(19) United States
(12) Patent Application Publication
(10) Pub. No.: US 2008/0280776 A1
(43) Pub. Date: Nov. 13, 2008

(54) Method and apparatus for detection of molecules using a sensor array

(76) Inventors: Rashid Bashir, Champaign, IL (US); Oguz Elibol, West Lafayette, IN (US); Pradeep Ramachandran Nair, West Lafayette, IN (US); Donald Bergstrom, West Lafayette, IN (US); Ashraf Alam, West Lafayette, IN (US); Bobby Reddy, JR., West Lafayette, IN (US)

Correspondence Address: BARNES & THORNBURG LLP, 11 SOUTH MERIDIAN, INDIANAPOLIS, IN, 46204, US

(21) Appl. No.: 11/999,215

(22) Filed: Dec. 4, 2007

Related U.S. Application Data
(60) Provisional application No. 60/868,387, filed on Dec. 4, 2006.
(60) Provisional application No. 60/972,528, filed on Sep. 14, 2007.

(51) Int. Cl. C40B 30/04; C40B 50/18; C40B 60/12; C12N 1/06; G01N 33/48; C12P 19/34; H05B 3/02

(52) U.S. Cl. 506/9; 506/32; 506/39; 435/259; 435/40.5; 435/91.2; 219/201; 435/286.1; 435/287.2; 435/6

(57) ABSTRACT

Apparatus and methods for detecting molecules in a fluidic environment are provided, including nanodevices and methods for fabricating, functionalizing, and operating such nanodevices. At least one of the methods includes selective heating of nanodevices in an array.

### RELATED APPLICATIONS

This application claims the benefit of U.S. Provisional Patent Application No. 60/868,387, filed Dec. 4, 2006, and U.S. Provisional Patent Application No. 60/972,528, filed Sep. 14, 2007, both of which are incorporated herein by this reference in their entirety.

### BACKGROUND

The ability to simultaneously detect multiple analytes in low concentrations in real time is of interest for a wide variety of biological diagnostics platforms, from DNA or protein assays to analyte-specific sensors. The ability to immobilize and pattern a large number of distinct probe biomolecules with precise spatial control is a desirable characteristic of functional sensors.

For example, biosensors can be used in the diagnosis of cancer and other diseases and conditions in which specific molecules or markers, such as proteins, may be detected in blood or other biological fluids. Ultra-sensitive detection of these markers at low concentrations can lead to early diagnosis, which can often result in a successful treatment for the disease. Often, there is more than one type of marker that provides information that can lead to a diagnosis. In these instances, the ability to detect a large variety of biomolecular markers simultaneously is desirable.

Known label-free methods and electrical techniques for detecting biomolecules include surface plasmon resonance (SPR), acoustic sensors, calorimetric sensors, electrochemical methods, and certain field effect devices. Semiconductor field effect sensors have been shown to enable the possibility of realizing label-free sensors for detecting chemical and biological species. In particular, field-effect devices realized using nanowires or similar materials and structures have been shown to provide better detection sensitivity and selectivity than other clinical alternatives. However, such devices have thus far not been able to be fabricated in large quantities. In addition, they have not been able to be functionalized individually, and have been prone to false positives due to non-specific binding of analytes.

A label-free cost-effective, high-throughput, sensitive, selective and portable biological sensor is desired. Currently known methods and devices may satisfy some of these requirements, but none are known that satisfy all of these requirements.

There is therefore a need for methods and techniques for developing reliable biomolecule sensors that are cost effective without compromising sensitivity or selectivity, that enable simultaneous multiple analyte detection, and which reduce the non-specific binding of molecules.

Such technologies are considered likely to have positive implications for disease diagnostics, drug discovery, and improving our understanding of cellular systems.

### SUMMARY

This disclosure describes methods to fabricate field effect devices on a large scale and to individually functionalize each device using localized heating. These methods may be used to create densely integrated, highly sensitive and selective biomolecule sensing devices that can be used for label-free detection of a wide variety of biomolecular species simultaneously in real time. In addition, devices created according to the disclosed methods may be integratable into existing sensor platforms.

In one embodiment, a method of forming a sensor array having a number of sensors configured to detect at least one target molecule in a liquid analyte is provided. The method includes exposing the sensor array to a liquid containing a probe molecule, applying, in the presence of the liquid containing the probe molecule, an AC signal to one sensor of the sensor array to heat the one sensor, and binding the probe molecule to the one sensor.

Each sensor may include a nanodevice. Each nanodevice may include a semiconductor, an insulator, at least one metal contact, and a microfluidic component. The nanodevice may be fabricated by a top-down, CMOS-compatible process. The nanodevice may be fabricated by a bottoms-up self-assembly technique. The nanodevice may be fabricated using a bonded Silicon on Insulator wafer using a Separation by Implantation of Oxygen technique.

A first liquid may be used to apply a first probe molecule and a second liquid different than the first liquid may be used to apply a second probe molecule different than the first probe molecule. The exposing, binding and applying steps may be repeated to form a sensor configured to detect a second target molecule different than the target molecule. The exposing, binding and applying steps may be applied to a first sensor in the sensor array to configure the first sensor to detect a first target molecule and the exposing, binding, and applying steps may be applied to a second sensor in the sensor array to configure the second sensor to detect a second target molecule different than the first target molecule.

A first AC signal having a first voltage may be applied to a first sensor in the sensor array to configure the first sensor and a second AC signal having a second voltage different than the first voltage may be applied to a second sensor in the sensor array to configure the second sensor. The applying step may heat a first sensor in the sensor array to a first temperature value and may heat a second sensor in the sensor array to a second temperature value different than the first temperature value to configure the sensor array. The AC signal may be applied to the sensor between an electrode and a bottom backgate of the sensor.

In another embodiment, a method of detecting at least one target molecule in a liquid analyte with a sensor array having a number of sensors is provided. The method includes exposing the sensor array to a liquid containing a probe molecule, applying, in the presence of the liquid containing the probe molecule, an AC signal to one sensor of the sensor array to heat the one sensor, binding the probe molecule to the one sensor, exposing the sensor array to the liquid analyte, applying an AC signal to the one sensor of the sensor array to heat the one sensor and the probe molecule, and detecting the at least one target molecule with the probe molecule, if the target molecule is present in the liquid analyte. The sensor array may be formed according to the above method.

In another embodiment, a molecule detection apparatus is provided. The apparatus includes a nanoplate sensor device made by the first method above, and computer software executable to perform the second method above. The apparatus may include a function generator coupled to the nanoplate sensor to provide an electrical signal to a sensor of the nanoplate sensor device. The electrical signal may be a sinusoidal signal. The apparatus may include a switch matrix coupled to the sensor and to the function generator to enable simultaneous measurement of multiple sensors. A cancer screening system including the above apparatus may be provided. Moreover, an miRNA screening system including the above apparatus may be provided.

In another embodiment, a method of selectively heating individual sites in a nanosensor array including a plurality of nanosensors is provided. The method includes determining a desired pitch length between sensors in the array, arranging the sensors in the array according to the desired pitch length, individually addressing each sensor site in the sensor array, determining a desired temperature for a sensor site in the array, and selectively increasing the temperature of the sensor site to the desired temperature by applying an AC voltage to the sensor site. The above method may be used for performing a temperature acceleratable chemical reaction, or for lysing cells. The method of lysing cells may be used to detect intracellular proteins. The determining and selectively increasing steps may be repeated to heat the sensor site to a different desired temperature. The above method may be used to perform a polymerase chain reaction at an individual sensor site. The above method may also be used to change surface properties of a nanosensor device.

Patentable subject matter may include one or more features or combinations of features shown or described anywhere in this disclosure including the written description, drawings, and claims.

### BRIEF DESCRIPTION OF THE DRAWINGS

The detailed description of the drawings refers to the following figures in which:

FIG. 1 is a simplified schematic of a nanowire sensor device, including a magnified view of a portion of the device;

FIG. 2 shows simplified cross-sections of fabrication steps for a sensor device according to FIG. 1;

FIG. 3 is a top view of a chip layout for a sensor device according to FIG. 1;

FIG. 4 shows a Transmission Electron Microscope (TEM) image of a cross-section of a nanowire for a sensor device (A), and Field Emission Scanning Electron Microscope (FESEM) images of a top view of a nanowire of a sensor device according to FIG. 1 (B)-(C);

FIG. 5 is an optical micrograph of nanowires in a sensor device according to FIG. 1;

FIG. 6 is a cross-sectional TEM image of a nanowire after completion of a fabrication process for a sensor device according to FIG. 1;

FIG. 7 shows simplified cross-sections of a nanoplate sensor device after various fabrication steps;

FIG. 8 is an AFM image of a representative nanoplate device;

FIG. 9 is a top view of a mask layout for a nanoplate chip incorporating nanoplate devices with independent source/drain contacts and devices with a common source and individual drain contacts, including a magnified view of a portion of the chip;

FIG. 10 shows optical microscope images of a field effect nanoplate device coated with a liquid crystal with a clearing point of 35 degrees Celsius after heating with different applied DC voltages;

FIG. 11 is a simplified cross-section of a nanoplate device showing application of alternating current;

FIG. 12 shows optical microscope images of a nanoplate device with a coating of liquid crystal with a clearing point of 60.5 degrees Celsius after heating with different applied AC voltages;

FIG. 13 shows graphical representations of fluorescently labeled DNA molecules applied to a nanoplate device to illustrate selective functionalization of the device using AC heating;

FIG. 14 shows fluorescent images of nanoplate devices before and after selective AC heating;

FIG. 15 shows fluorescent images of nanoplate devices before and after AC heating at different applied voltages;

FIG. 16 shows images and schematics of nanoplate devices having different oxide films at different regions having varying relative attachment efficiencies;

FIG. 17 shows schematics illustrating a heat catalyzed exchange reaction;

FIG. 18 is a schematic of a data acquisition system for sensing using a nanodevice for molecule detection;

FIG. 19 is a simplified schematic of a platform for a nanosensor array, including a magnified view of a sensor site;

FIG. 20 is a simplified schematic of an integrated lab on a chip usable for detection of cancer proteins and markers, including a nanosensor array;

FIG. 21 is a simplified schematic of an apparatus for laser-mediated site-specific heating including a nanosensor array;

FIG. 22 is a representative schematic of a disposable sensor cartridge including a nanosensor array; and

FIG. 23 is a perspective view of an embodiment of a portable screening system including a measurement reader and a nanosensor cartridge.

### DETAILED DESCRIPTION

This disclosure refers to illustrative embodiments of methods and apparatus for detecting molecules using a sensor array, which are shown in the accompanying drawings and described herein.

### Nanowire Sensor Device and Fabrication Method

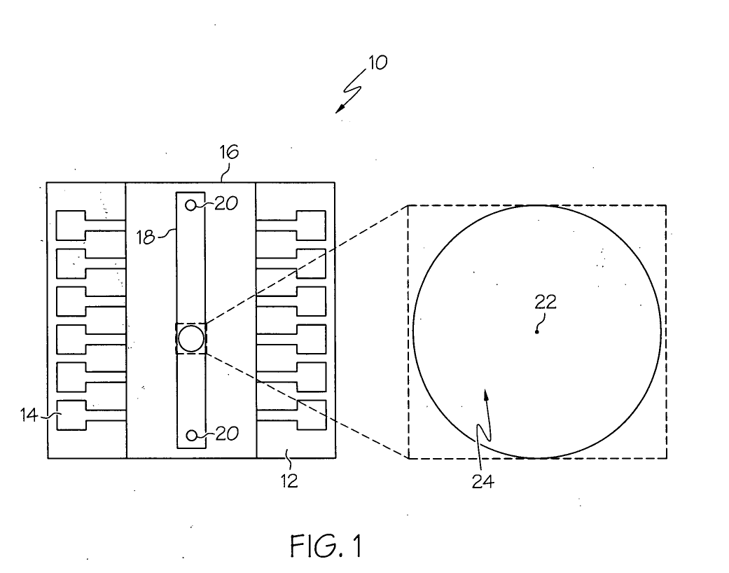

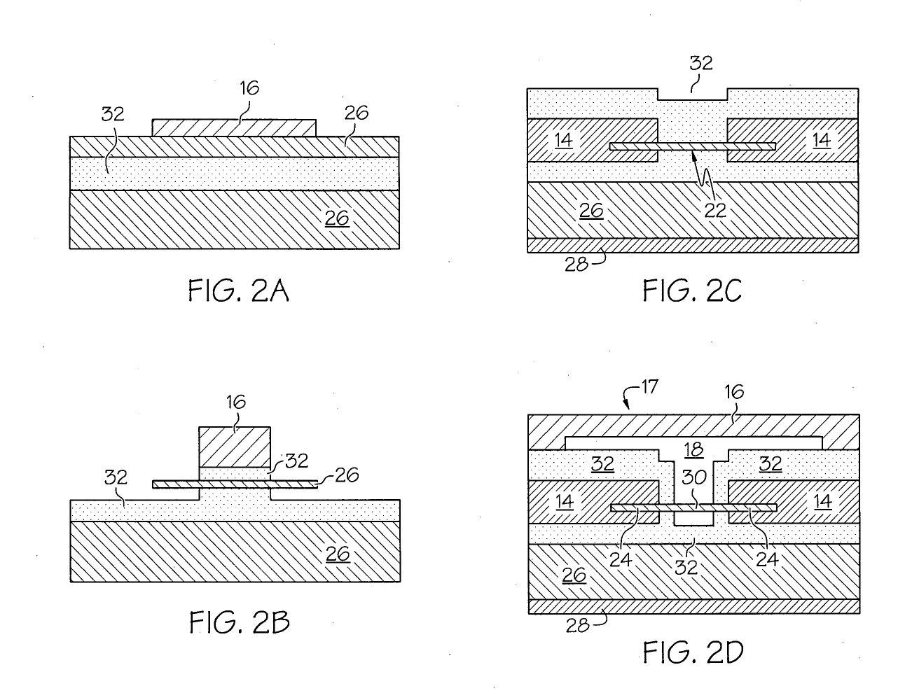

In one embodiment, a nanowire sensor device is provided. FIG. 1 schematically shows a top view of an illustrative nanowire device 10. The device 10 generally includes a substrate 12, a plurality of spaced apart, independently addressable electrical contacts or pads 14, a cover 16 defining a fluidic channel 18 having input and output regions 20, and a nanowire sensor 22. Nanowire sensor 17 includes a plurality of spaced apart nanowires, with each nanowire having a first and second end portions 24 coupled to opposing contacts 14, and a middle portion 30 positioned in channel 18, as best shown in FIG. 2(d).

The substrate 12 includes a silicon wafer 26 and a back side contact 28 as shown in FIG. 2(d). The initial wafer has a top silicon layer thickness of about 50 nm and a buried oxide thickness of about 160 nm. The electrical contacts 14 are patterned to measure the conductance of the nanowires at regions 24. The cover 16 comprises a silicone elastomer such as poly-dimethylsiloxane (PDMS).

In operation, an analyte is introduced into channel 18 via an input region 20, thereby causing a change in the overall resistance of the nanowire 22, which can be sensed externally by monitoring the nanowire conductance.

The fabrication of the device 10 begins with a silicon wafer such as a SIMOX SOI wafer (Separation by IMplantation of OXygen Silicon On Insulator), having a top silicon layer thickness of about 50 nm and a buried oxide thickness of about 160 nm. Next, lines that are about 50 nm in width and many microns long are defined by electron beam lithography (JEOL 6000FS) using a negative e-beam resist (NEB-31), as shown in FIG. 2(a). Patterns are transferred onto the silicon via chlorine based reactive ion etching, and the e-beam resist is removed after this step. This results in silicon wires with nearly square cross-sections measuring about 50 nm on each side.

Next, the wafer undergoes a lengthy (about 9 hours) oxidation step performed at 950±C. Since the oxidation temperature used is below the glass transition temperature of the oxide, the formed oxide does not reflow, causing a stress buildup at the silicon-oxide interface. As the oxidation continues, this stress increases with increasing oxide thickness, and due to the dependence of the oxidation rate on the stress at the interface, the oxidation self-terminates, leaving a silicon core at the center of the structure.

The core cross-sectional size and shape can be fine tuned with careful control of parameters such as starting wire diameter, cross-sectional geometry and oxidation temperature. Using this technique it is possible to obtain sub-20 nm diameter silicon wires reliably and reproducibly. After this step, optical lithography is performed to define the metal layer. Before metal evaporation the oxide above the source and drain regions of the wire is etched to enable electrical contact to the wire, as shown in FIG. 2(b).

Liftoff is performed after the evaporation of the metal. Next, the metal layer is isolated from the fluidic environment by coating a layer of Plasma Enhanced Chemical Vapor Deposited (PECVD) oxide on the metal, as shown by FIG. 2(c). The nanowires are exposed to the environment by etching the oxide directly over the wire. A PDMS layer or cover 16 containing the microfluidic channel 18 caps the fabricated chip for delivery of fluid to the nanowire sensors as shown by FIG. 2(d).

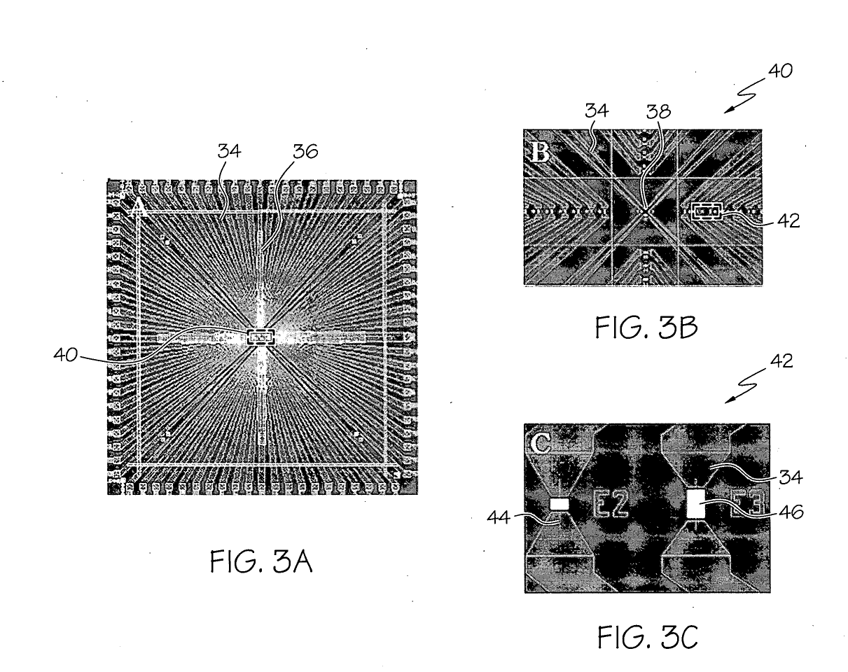

FIG. 3(A) shows a top view of a chip layout for device 10 including an array of nanowire sensors. The completed chip is about 1.5 cm by about 1.5 cm in size, and contains 84 pads arranged around the perimeter of the chip. The chip is designed to fit to an 84 pin LCC package. The portions 34 represent the metal layer, tracing from the outside of the chip to the center, where the silicon nanowires are located. This design allows for the individual addressing of 40 nanowires, along with control of the fluid potential at 4 different locations.

Also shown in FIG. 3(A) is the microfluidic channel 36, which is placed on the chip for analyte delivery. The fluidic channel is substantially cross-shaped as shown in the figure, allowing fluidic ports to be placed at each end of the cross. Depending on the configuration of the inlets and outlets, this design allows the fluid to stagnate in certain channels while enabling fluid flow in other channels.

FIG. 3(B) depicts a magnified view of area 40 of FIG. 3(A), i.e., the center of the chip. Each metal line 34 terminates at a terminal of a nanowire. The nanowires 38 are placed in the fluidic channel 36 in a substantially cross-shaped configuration, located at the centerline of the fluidic channel 36.

FIG. 3(C) shows a magnified view of area 42 of FIG. 3(B), i.e., the placement of individual nanowires 44 relative to metal layers 34. FIG. 3(C) also shows the sections 46 in which an analyte contacts the nanowires 44 when the sensor is in operation.

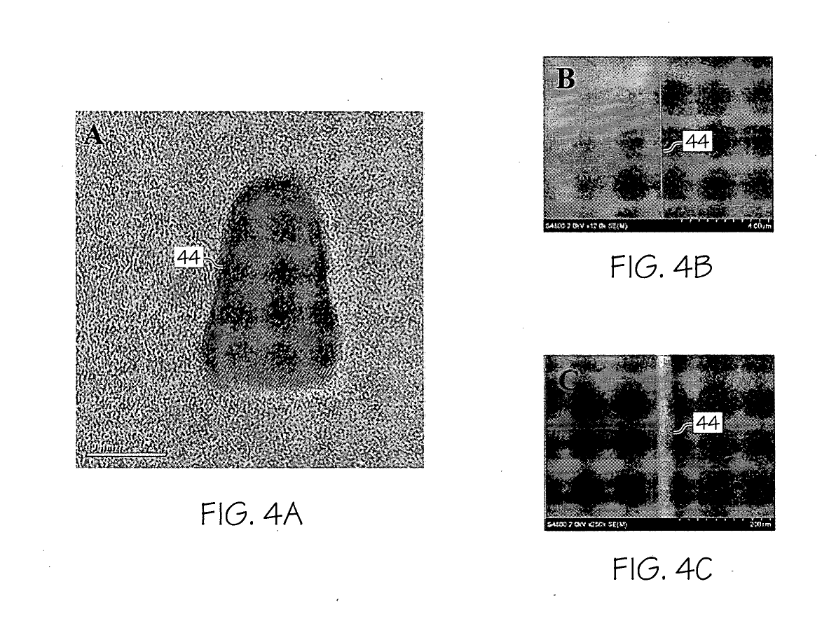

FIG. 4(A) shows a top view, and FIGS. 4(B) and (C) show cross-sections of nanowire 44. FIG. 4(A) is a high resolution TEM (Transmission Electron Microscope) image of the cross-section of the fabricated silicon wire encapsulated in oxide, with the largest dimension of about 24 nm in the vertical direction, and about 15 nm in the horizontal direction, resulting in an effective diameter of about 19 nm (by cross-sectional area). The TEM image also shows the high crystal quality of the wire, with no defects or dislocations.

FIG. 4(B) shows an FESEM (Field Emission Scanning Electron Microscope) image of a top view of the silicon nanowire 44, with some of the oxide encapsulating the wire partially etched. The wire is substantially continuous and uniform in diameter for the entire length. FIG. 4(C) is a magnified view of FIG. 4(B), showing the substantially smooth edges.

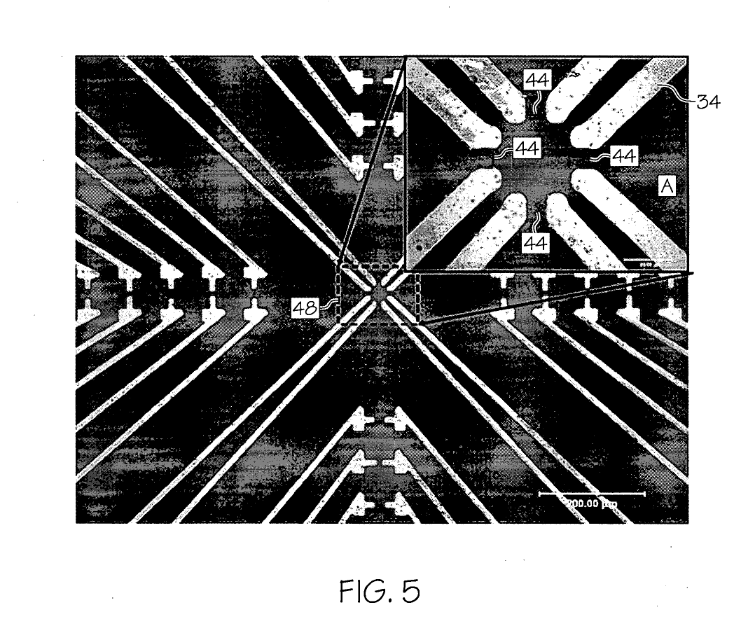

A typical optical micrograph of the completed device is shown in FIG. 5. The inset (A) shows a magnified view of box 48, i.e., the center of the die with metal contacts 34 independently contacting nanowires 44.

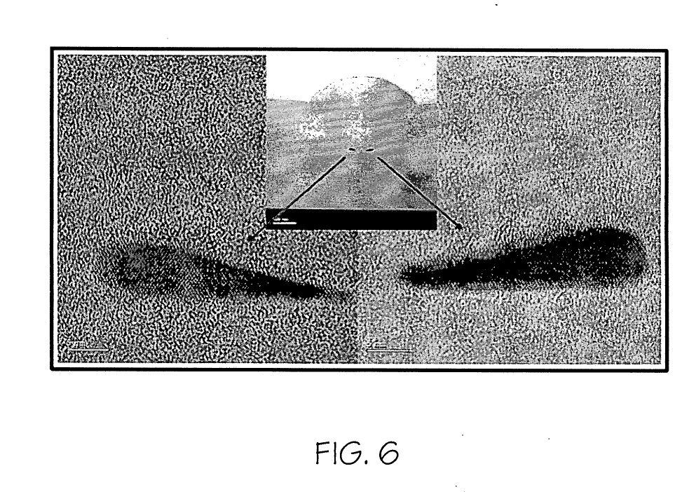

As shown in FIG. 6, after application of the oxide layer, further magnification reveals that the resulting wires 44 are actually two wires instead of one, and the shape of the wires is different than wires of known fabricated devices. The initial width of the wire is estimated by e-beam lithography to be around 200 nm. This yielded self limiting oxidation behavior only at the edges of the mesa, ultimately yielding tear-drop shaped wires. The self oxidation behavior is related to the radius of curvature of the oxidized region, which is very low only at the corners. Towards the center of the mesa, the radius of curvature approaches infinity, and the oxidation rates will approach those observed for planar oxidation.

Functional nanowires may therefore be reliably obtained using optical lithography, since nanowires can be formed at the edges of the mesas, regardless of the width of the mesa. Due to the increased complexity of utilizing electron beam lithography over standard optical lithography in fabricating nanowire structures, a device with the same thickness but a larger width may also be used.

The above fabrication process for realization of single crystal silicon nanowire structures is a CMOS compatible process. A combination of electron beam lithography and self limiting oxidation reliably yields substantially defect free nanowires with diameters of down to 20 nm. The process was later demonstrated to work for the fabrication of devices at the full wafer scale, particularly for devices with comparable thicknesses but larger widths.

### Nanoplate Sensor Device and Fabrication Method

In another embodiment, nanoplate sensor devices are used to realize a highly sensitive transducer. These devices are similar to the nanowire devices in that they may be fabricated using a Silicon On Insulator (SOI) wafer with an ultrathin silicon layer in the range of 10-25 nm, and they have a in the range of tens of microns. However, the nanoplate devices have a larger width, being about 2 μm in width.

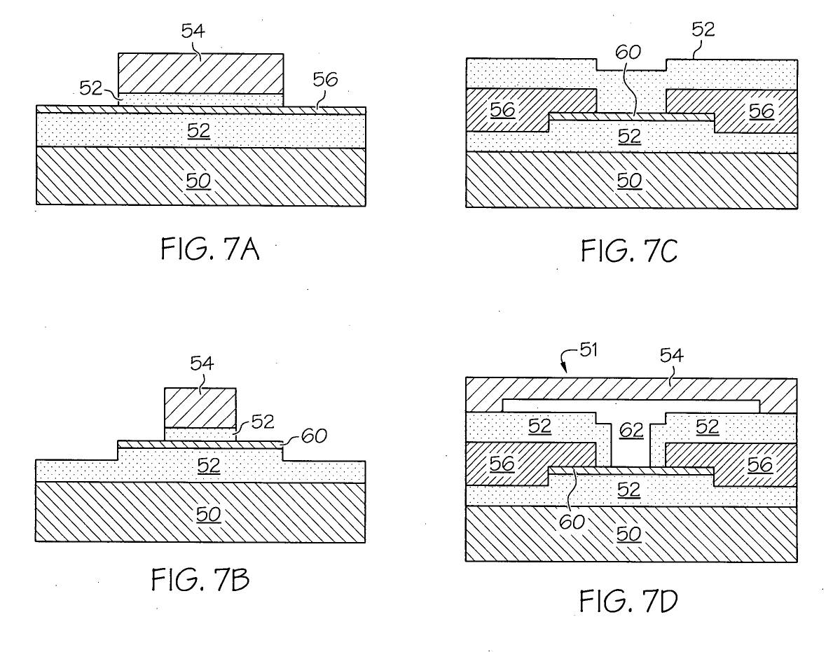

FIG. 7 schematically depicts cross-sections of a nanoplate device 51 after various fabrication steps. The completed nanoplate device includes a substrate or wafer 50, a first oxide layer 52, source/drain and drain/source contacts 56, second oxide layer 52, a cover 54 defining a fluidic channel 62, and a nanoplate 60.

Fabrication begins with a 4 inch SIMOX (Separation by IMplantation of OXygen) or Bonded SOI wafer. The wafer has a top silicon layer 56 of about 50 nm and a buried oxide layer 52 of about 400 nm, with p-type doping. Suitable wafers may be purchased from SIMGUI electronics, of Shanghai, China.

The superficial silicon is thinned down to about 10 nm via wet oxidation performed at 900±C for about 14 min. The silicon active area is defined by lithography, and the field oxide is etched using buffered oxide etchant (BOE) as is shown in FIG. 7(a). Silicon in the field area is wet etched for about 90 seconds using a Tetra-Methyl Ammonium Hydroxide (TMAH) solution heated to about 60+C. An implant mask defined by lithography is used to implant dopants only in the contact areas.

Boron is implanted at 25 KeV at a dose of 1014 cmᵢ2. The implant mask was stripped off and the dopant activation was performed at 1000±C for 2 min in a rapid thermal annealer (RTA). Contacts to the active area are defined by lithography utilizing a liftoff process. Before the metal evaporation, silicon dioxide over the contact regions is etched for about 70 seconds using BOE as shown by FIG. 7(b). An adhesion layer of 200° A of titanium followed by a 1800° A layer of platinum is evaporated to form a metal contact. After the liftoff process, a rapid thermal anneal (RTA) is performed at about 500 C. for 60 seconds in order to improve the quality of the metal contact to the silicon.

As shown by FIG. 7(c), Plasma Enhanced Chemical Vapor Deposited (PECVD) oxide is deposited as a metal passivation layer, in order to minimize the parasitic conductance through the fluidic environment. Oxide is etched directly over the pad areas, and a thick layer of metal (about 2000° A titanium and about 8000° A of gold) is evaporated and defined by liftoff to form pads for wire bonding.

Next, areas over the active area of the devices are defined by lithography. The wafer is then diced into individual dies of size of about 4 mm by 7 mm. Individual dies are then etched using BOE to expose the active area of the devices. A master mold defined on a silicon substrate with about a 25 micron thick SU8 layer is used to create the PDMS microfluidic channels, which are then bonded to the chips as shown by FIG. 7(d).

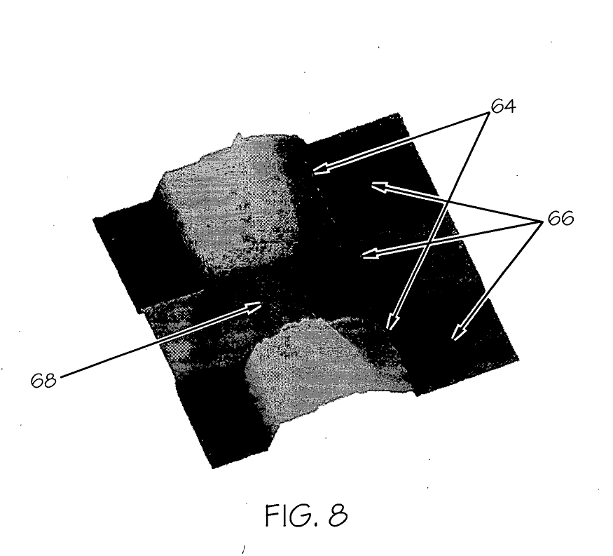

FIG. 8 shows an STM image of an embodiment of a nanoplate device after electrode definition. The design consists of 6 mask levels: 1-Active Area Definition; 2-Implant Mask; 3-Metal Definition; 4-Pad Etch; 5-Release Window; 6-Microfluidic Channel. The illustrated embodiment includes metal electrodes 64, an oxide substrate 66, and a silicon or semiconductor active area 68 and has an active area of about 10 um in length, 4 um in width and 30 nm in thickness. It will be understood by those skilled in the art that the height differential shown in FIG. 8 is not real.

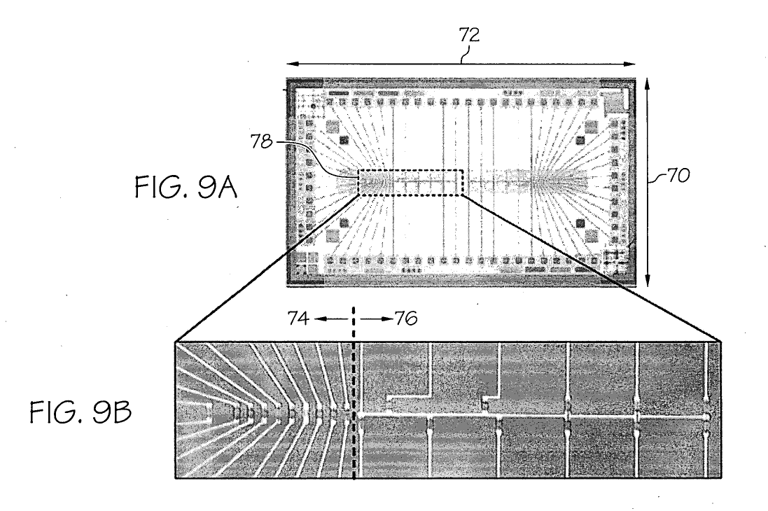

FIG. 9(A) shows an example of a completed die for a nanoplate device having a first dimension 70 and a second dimension 72. In the illustrated embodiment, the mask set results in 243 complete dies that span a 4 inch wafer, with a first dimension 70 of about 4 mm and a second dimension 72 of about 7 mm on each side. The chip size was chosen to fit a 24 pin DIP package.

In the array, there are 20 devices with separate source and drain contacts, and 2×11 devices with a common source and individual drain contacts, as shown by FIG. 9(B), which is a magnified view of the area inside box 78 of FIG. 9(A). FIG. 9(B) shows an independent source/drain portion 74 and a common source portion 76.

Devices with no active area in between the contacts and devices with the active area encompassed within oxide may be incorporated in the design as negative and positive controls. Various test structures may also be incorporated into the design, such as capacitors to extract C-V measurements, Van der Pauw structures to determine the sheet resistivity of the doped and undoped silicon layer, and stick resistors to determine contact resistance. Various other structures may be used to aid in the fabrication process, such as alignment marks, and large openings to measure film thicknesses.

An optimum temperature for performing the anneal may be determined, as increasing the temperature of the anneal beyond a certain temperature value may adversely affect the devices' electrical characteristics. For example, in the illustrated embodiment, performing the anneal at 600±C resulted in significant degradation of the maximum current through the device.

Although the general shape of the characteristics remains unchanged after RTA, the magnitude of the current increases significantly. Drain induced barrier lowering (DIBL) may occur, in which the source-drain electric field partially modulates the carrier concentration in the active area. This may cause higher currents for the positive source voltages, and lower current for negative source voltages.

After PECVD oxide deposition, the silicon layer inverts and predominantly electron conduction occurs. Larger current levels are seen for positive gate voltages, because of the further increase in the electron concentration. Also, higher currents are observed for negative source voltages and lower currents are observed for positive source voltages.

Upon completion of the fabrication process and obtaining individual dies, electrical testing may be performed while flowing fluid through the microfluidic channel. The current passing through the active area is modulated by the pH of the fluid due to changes in surface charge. As the pH of the solution increases, surface hydroxyl groups undergo deprotonation, increasing the negative charge on the surface. Hence for increasing pH values, the conductance is increased for a p-type device, and decreased for an n-type device.

A completed device may also be tested before flowing any fluid, while flowing DI water, while flowing phosphate buffer solutions at pH values of 5, 6, 7 and 8, and after drying the device. Under wet conditions current may be likely to leak from the source/drain regions through the BOX and into the back gate, although the same devices may have negligible conduction through the back gate under dry conditions. It is possible that the buried oxide may cause such leakage. Design considerations relating to the buried oxide are discussed below.

There are a number of design considerations for fabrication of the nanoplate sensors. For example, one choice of material for the metallization of the devices may be gold (Au), due to the limitations in available wire bonding materials and the requirement of using an inert metal that functions as an oxide etch stop. Furthermore, if the final bond pad material is gold, gold may be a reasonable choice for the first layer metallization. For example, 200 Å of Titanium as an adhesion layer and 2000 Å of gold as the contact material may be used. However, such use of gold may lead to undesirable effects after the deposition of PECVD oxide, such as debilitating cracks formed in the PECVD layer.

Another choice for the first metal layer may be platinum, due to inertness when faced with acids, and better adhesion to PECVD oxide. In general, the selected metal should be inert to the BOE performed to open up windows in the PECVD oxide before the pad metallization.

In order to form an ohmic contact to silicon, the silicon layer is heavily doped. This allows for the energy barrier between metal and silicon to become sufficiently narrow so that tunneling through the barrier becomes the primary carrier transport mechanism regardless of the metal-semiconductor work function difference.

There are two limiting cases for a metal contact on a semiconductor. In the absence of surface states the barrier height is mainly determined by the work function difference. If a large density of surface states is present at the semiconductor surface, then the Fermi level is pinned by the surface states and the barrier height is determined by the surface properties of the semiconductor. For Si the behavior of the barrier height is in between these two extremes.

The specific contact resistance depends strongly on both the barrier height and the doping concentration. For the Ti/Si system, the Schottky barrier height is around 0.6 eV; hence, the contact resistance is mainly a function of the doping level in the silicon.

The amount of active dopant in the silicon structure is a factor in lowering the contact resistance. Thus, a rapid thermal anneal (RTA) may be used since it provides higher dopant activation compared to furnace activation. The solubility of silicon for dopants increases with increasing temperature, and using RTA for the process allows the wafer to experience higher temperatures due to much shorter ramping times.

Buried oxide quality is a factor in determining the reliability and yield of SOI devices. Design concerns include the conduction through the buried oxide and carrier trapping in the buried oxide.

The buried oxide in SOI wafers manufactured via SIMOX may exhibit undesirable conduction characteristics both at bulk and localized levels. As the technology of fabricating SIMOX wafers matures, the severity of the defects should be reduced due to improvements in the preparation of the wafer before the implantation.

In addition to the potential defects associated with the buried oxide in the SIMOX devices, stress induced phenomena may occur. Due to enhanced stresses around the active area edges (isolated by a LOCOS process), there may be increased dopant diffusion, which in turn may affect the turn on voltage. As the perimeter to area ratio increases in such devices, the effect of dopant accumulation on the Silicon/Oxide interface on the threshold voltage of the device may become more pronounced.

As the superficial silicon layer thickness decreases, the surface to volume ratio of the active area increases, which is generally desirable for biosensors. Thinning the silicon layer leads to the domination of the bulk conduction properties by surface properties. This may be advantageous for the detection of a binding event at the surface; however, it may also be a disadvantage when interface traps between the silicon/buried oxide and the silicon/top oxide interface dominate the conduction characteristics used for detection.

Typical interface densities at the oxide/silicon interface may be on the order of 1010 to 1011 cmᵢ2, which is enough to completely deplete a 20 nm thick silicon layer at a doping of 1015. Thus, the bulk conductance is highly related to the surface properties. The conductance through a silicon-on-insulator membrane is significantly increased after obtaining an ultra-clean top silicon surface in vacuum. Therefore, the increase in conductance may be due to the formation of a surface layer made of dimer bonds which in turn interacts with the bulk silicon, effectively doping the bulk silicon with hole carriers.

The dimer bonds form bonding and anti-bonding surface energy states, and the thermal excitation of electrons out of the valance band of the bulk silicon into the almost empty bonding level creates free holes, pulling down the Fermi level. In this case there are two conduction paths, electron conduction through the surface, and hole conduction through the bulk silicon. However, the surface conduction mobility may be negligible, and conduction through the bulk in this case may dominate.

In sensor design, it may be desirable to establish an active area that is lightly doped to increase the sensitivity of the device, while at the same having enough carriers in the layer to make sensing practical. In view of this, a layer of designed molecules may be provided on the surface and selecting an appropriate band structure may be used to control the electronic transport properties of the bulk conduction layer. For example, for sub-50 nm thick silicon layers the amount of carriers in the bulk is comparable to the interface states; hence, the resistance of the layer steeply increases.

In the illustrated embodiment, the carrier concentration in the active area may be modulated using the back gate. However it may also be desirable to keep to gate voltage at the same potential at the source/drain contacts so that there is no electric field in the fluid that might affect the diffusion of any charged molecules.

An AC voltage may be applied between the top silicon layer and the substrate to produce a shift in the threshold voltage. Since the source and drain electrodes are shorted to each other, the bias is effectively applied between the silicon layer and the substrate. Applying higher voltages and higher frequencies may resulted in larger shifts in the threshold voltage. For example, a 12 MHz AC signal at an amplitude of about 40 Vrms may be applied for about 10 min., resulting in a threshold voltage shift of about 43V. Fine tuning of the threshold voltage to maximize sensitivity by manually shifting the IdᵢVg characteristics may be used to achieve maximum transconductance at a 0 V gate bias.

In the illustrated embodiment, nanoplate sensor devices are fabricated and tested that are about 10 nm thick, 2 microns in width, and 30 microns in length. These devices are very sensitive to surface charge, as deposition of PECVD oxide shifted the threshold voltage tens of volts.

In order to prevent current leakage from source and drain to the back gate, bonded SOI wafers may be used. Such bonded wafers may be fabricated by bonding a superficial silicon layer on a wafer with thermal oxide in order to reduce or eliminate defect issues that may occur with the SIMOX BOX. Further details of various fabrication methods, including a suitable fabrication method for bonded SOI wafers, are described in Elibol, Highly Sensitive and Selective Label-Free Nanoscale Sensors for Ultra Large Scale Integrated and Multiplexed Real Time Detection of Molecules: A Preliminary Report Submitted to the Faculty of Purdue University In Partial Fulfillment of the Requirements for the Degree of Doctor of Philosophy, May 2007, which is incorporated herein by this reference in its entirety.

The fabrication method to be used for site specific functionalization of devices, or local heating of devices, is not limited to CMOS compatible top-down fabrication methods discussed earlier. The disclosed devices may be fabricated using bottoms-up fabrication approaches. For example, the nanowires can be realized by using a vapor-liquid-solid (VLS) method and patterned on a substrate, forming contacts to the nanowires before or after patterning. Consequently, any electrically conductive parts can be passivated with an appropriate dielectric such as silicon nitride.

### Functionalization Methods

A method for the individual functionalization of nanoplate field effect devices via selective resistive heating is provided. This scheme is designed to produce dense arrays of nanoplate devices capable of ultra-sensitive detection of several biomolecular species simultaneously in real time with increased specificity.

The method involves heating of individual nanoplate devices. Individual devices can be heated by using either a direct current (DC) through the device, or by applying an alternating current(AC) between the device and the back gate.

A nanoplate device is fabricated according to a fabrication method described above. The device is individually functionalized, to enable simultaneous multiple species detection, using selective resistive heating of the surface of the channel via application of a voltage bias across the device. Heating an individual device allows for the partial disassociation of previously immobilized protection molecules on the active area, enabling the attachment of a probe targeting a specific biological species.

Another device can be similarly functionalized to allow for attachment of a different probe molecule targeting a different species. This allows for the specific placement of a wide variety of probe molecules to only specifically selected sites, which in turn allows for individual functionalization of the wires in a dense array. The composition of a fluid containing several of these biomolecular species at extremely low concentrations can thus be determined in real time.

According to the disclosed method, heating characteristics of the sensors are determined in order to perform selective functionalization. The temperature attained on the surface of the nanoplate devices can be characterized as a function of the power input to the devices by using temperature sensitive nematic phase liquid crystals. Suitable liquid crystals include those available from Accelerated Analysis.

When the devices are heated by applying a current through the active area, power is dissipated through the voltage drop across the active area, resulting in an increase in the surface temperature. The power dissipation is correlated with the surface temperature of the devices.

The devices may be solvent cleaned (e.g., using Acetone and Methanol) prior to the application of the temperature sensitive liquid crystals (LCs). Liquid crystals, of various clearing temperatures are applied on the device for each individual experiment using a fine tip paint brush. The LC dissolves readily in a solvent (as purchased), and upon the application of the liquid crystal, the solvent evaporates leaving a thin layer of coating.

Device leads are contacted by using a microprobe manipulator on the source and drain sides, and the back gate (substrate) is contacted through a conductive chuck. The chip surface was imaged via a microscope to observe the change in the intensity of the liquid crystal. A 100× objective is used to image the individual devices. Images may be acquired using a commercial digital still camera (Canon G5) attached to the microscope and connected to a computer via a USB cable. Remote Capture software may be used to capture pictures automatically at preset time intervals. A Labview program (National Instruments) on this computer may be used to synchronize the capture of pictures with data readouts from the DC power supply (Agilent E3647A), the low noise current amplifier (SRS SR570), and a digital multimeter (DMM)(HP 3478A) via GPIB and RS232 protocol. The DC power supply may be used to supply the source-drain and gate-source bias (with two independent outputs), and the current amplifier in conjunction with the DMM may be used to measure output current through the device. The gate voltage is set to a bias to modulate conduction through the device to a reasonable level. The source-drain voltage is swept at a rate of 0.1 V per second, typically in a range of 0-40 V. MATLAB may be used to plot the intensity over a section of the active area versus input power for the device.

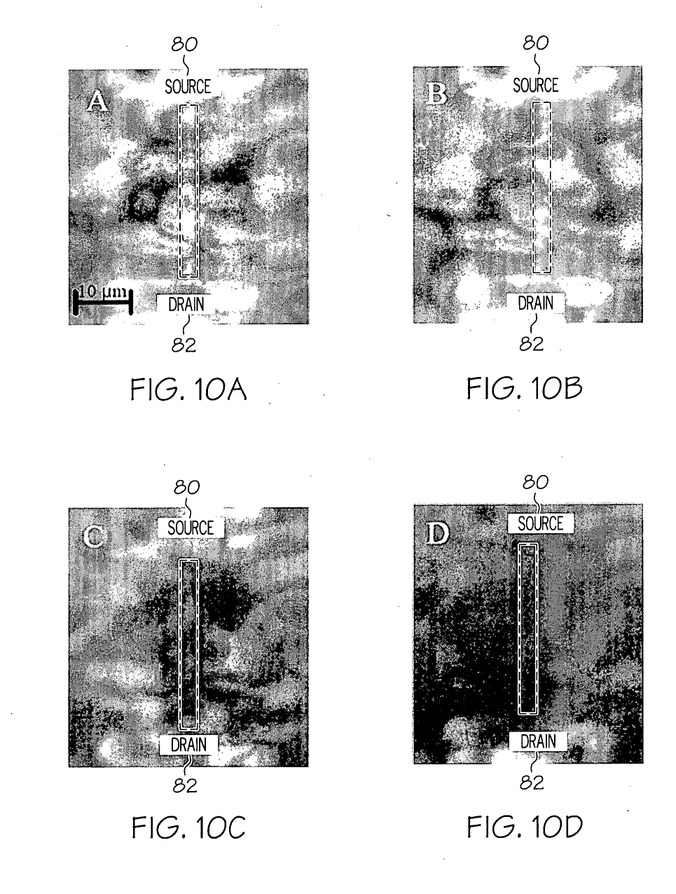

FIG. 10 shows optical microscope images of an embodiment of a field effect nanoplate device having a source 80 and a drain 82. The device of FIG. 10 is coated with a liquid crystal having a clearing point of 35° C. The reflected light intensity over the channel region decreases sharply as the active area heats up, allowing the characterization of temperature on the nanoplate as a function of input power.

In order to quantify the surface temperature, the average intensity over the active area may be plotted as a function of the input power. The transition power for the given temperature is determined by reading the power corresponding to the inflection point of the fitted intensity versus power curve. Similar characterization may be completed with different LCs of varying clearing temperatures, such as −29° C., 35° C., and 60° C. The objective is to determine the temperature differential that can be achieved (not the absolute temperature).

Temperature on the surface can be quantified with a reasonable amount of error using the above techniques. The amount of power needed to attain a certain temperature on the device surface can thus be quantified using this method. Any effect of the electric field on the liquid crystal properties is negligible as the clearing temperature is shown to be consistent.

Through experimentation further described in Elibol, incorporated by reference above, it has been determined a temperature differential of 14° C. is achievable with the above described nanoplate devices, and that this temperature differential is expected to be more than adequate to perform the exchange reactions necessary to individually functionalize several of the devices.

Once selective functionalization of individual sensor elements in an array has been achieved, charge states of mismatched genomes must be successfully differentiated, because two genomes of the same length have essentially the same integrated charge. Hence techniques which can filter out non-specific binding are desirable for electrical detection schemes. One way of accomplishing this is by exploiting the difference in the melting temperatures of such sequences.

A small ¢TM for some base-pair mismatches indicates the possibility of non-specific binding in a nano-sensor array. As such, the weakness of the binding energy of the conjugation may be used to distill the array of parasitic/non-specific binding. Many parasitic bindings can be filtered out if the temperature profile around the binding site can be accurately controlled. As described herein, selective heating of nanoplate devices can be used to achieve accurate control of temperature profiles of individual sensor elements. This capability is useful in filtering out such non-specific target-receptor bindings and thus is a desirable element in enabling highly selective, real time, and multiple species detection in a sensor array.

Using temperature sensitive liquid crystals with resistive heating of the disclosed fabricated nanoplate devices, a temperature differential of up to 14° C. can be achieved. The fabrication process may be calibrated to allow for larger achievable temperature differentials by DC heating.

The specific heating of the nanoplate devices allows exchange reactions to be performed at only the desired selected sites, thereby providing targeted functionalization of the devices. The top-down fabrication process described above scales, allowing for relatively seamless integration with traditional electronics. Additionally, the disclosed method of precise temperature control of our individual devices is designed to eliminate nonspecific binding. The end result is a large array of devices targeted towards the highly specific, ultra-sensitive detection of a large variety of biomolecular species.

DC heating of the devices may yield to unwanted electrophoretic transport of analytes, and with increased bias may even lead to the electrical breakdown of the surrounding passivation layer. A scheme in which heating can take place without a large difference in the bias of the connecting electrodes may therefore be desirable.

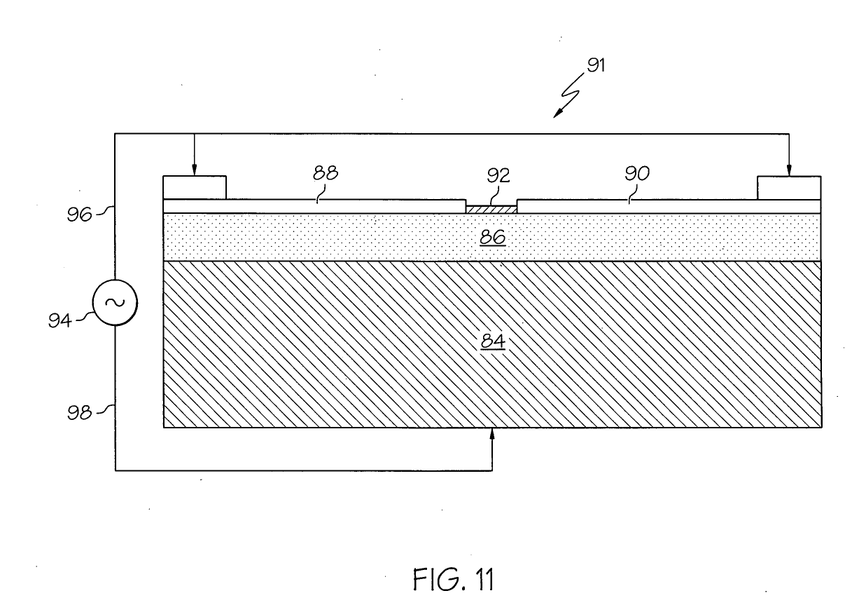

FIG. 11 shows a nanoplate device 91 with alternating current being applied. The illustrated device includes a silicon substrate 84, buried oxide 86, a source contact 88, a drain contact 90 and an active area 92 between the source and drain contacts 88, 90. An alternating current source 94 is applied via electrical signal conduits 96, 98. In the illustrated embodiment, the chip is placed on a brass plate that functions as a chuck to form the back contact. Micromanipulator probes are used to contact the appropriate pads on the chip. The source and drain contacts are shorted and an AC bias is applied between the source/drain and the back contact as illustrated in FIG. 11.

A functionalization method including applying an alternating current between the top electrodes and the bottom backgate of the nanoplate device is also provided. The method includes electrically shorting the source and drain regions to form a single top electrode as shown in FIG. 11. Applying an AC voltage through the dielectric layer between the top electrode and the bottom electrode then results in dielectric relaxation. This leads to energy dissipation and hence heating in the dielectric layer. This heat is dissipated through the bulk silicon and the top metal electrodes, which both act as a heat sinks. This also results in heating of the active area of the devices.

Similar to the characterization of DC heating on the nanoplate devices, liquid crystals are used to characterize heating attained by using the AC heating scheme. In the illustrated embodiment, a liquid crystal with a clearing temperature of 29±C is coated on the nanoplate device. The device is placed on an external heater with an external RTD connected to the heater to sense the ambient temperature. Individual devices are heated by applying an AC voltage in the range of about 10 MHz to 15 MHz and in the amplitude range of about 0 to 7 V. The voltage and temperature to attain a certain temperature increase are characterized. The temperature increase may be observed over an area of approximately 1 mm².

Through experimentation described in Elibol, incorporated by reference above, it has been determined that the observed heating is due to dielectric heating, and thus can be used for heating individual devices for performing selective functionalization.

Heating of individual devices may be tested using the LC technique described previously. Similar to the procedure outlined above, the liquid crystal may be applied to the chip, and still photographs may be acquired at different input voltages. Results may be obtained by analyzing the data using MATLAB. An AC voltage may be applied using a function generator connected to a 50 dB RF power amplifier.

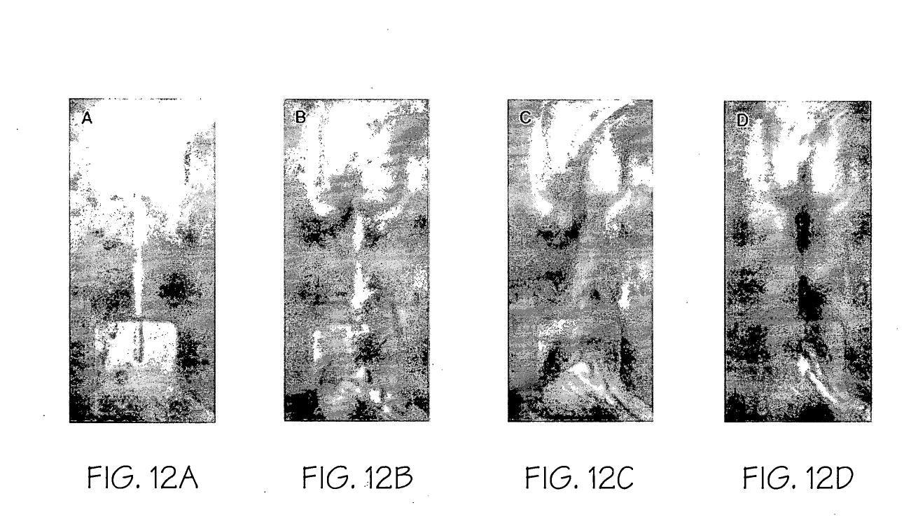

FIG. 12 shows a close up of a nanoplate device coated with a 60.5±C clearing point LC (Ambient temperature at 21±C) at different applied AC voltages. An AC bias at a frequency of 100 KHz is applied at various voltages: 0 V in image (A), 5.6 V in image (B), 11.5 V in image (C), and 17.5 V in image (D).

Heating of individual devices may therefore be used as a means for individual functionalization of nanoplate devices on the chip. As an example of the application of the disclosed method, desorption of fluorescently labeled DNA molecules on nanoplate devices is described.

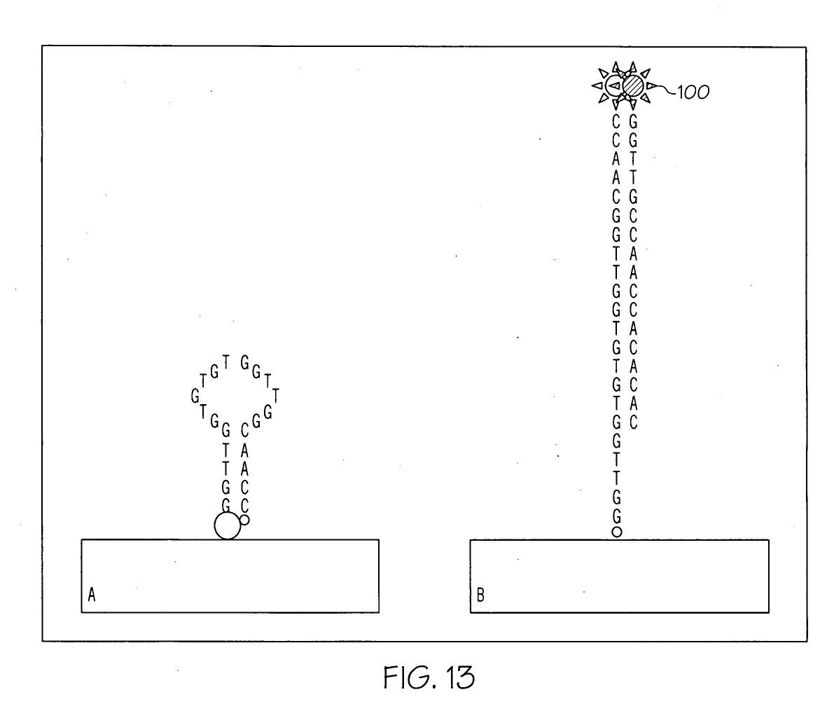

In this example, adsorbed species are successfully removed from individual devices. A molecular beacon with fluorescein is covalently immobilized on the chip surface. A molecular beacon is a hairpin loop structure that has been modified with both a fluorophore and a quencher that under normal conditions quenches the fluorophore. FIG. 13 (a) shows a representation of the molecular beacon modified surface in a closed configuration.

When the hairpin loop is opened, the fluorescence is restored, indicating the presence of the complementary strand. Later, a complementary DNA modified with a ROX(red) fluorophore is introduced to open the hair pin loop. FIG. 13(b) shows the ROX modified complementary strand 100 attached to the molecular beacon. The change in intensity of the fluorescence may be studied upon heating of the individual devices.

The hairpin loop may be purchased from Sigma-Genosys (5′-CCAACGGTTGGTGTGTGGTTGG3′) with the 3′ end modified with an amine group as well as an internal quencher (DABCYL). The 5′ end may be modified with fluorescein.

The molecular beacon is covalently immobilized on an amino-silanzied surface using a homobifunctional crosslinker. Samples may be cleaned with a Piranha solution (H2O2: H2SO4)(3:1), rinsed with DI water, then dried using a stream of nitrogen immediately before the start of the chemistry. Subsequent steps may be performed in a glove box purged with high purity nitrogen. Samples may be silanized in a 3% 3-aminopropyltrimethoxysilane (purchased from Sigma) in a Methanol:DI(19:1) solution for 30 minutes at room temperature. Subsequently, the samples are rinsed with Methanol and DI water, and dried with nitrogen. Later, chips are cured using a hot plate at 110±C for 15 minutes. Chips are placed into a dimethylformamide (DMF) solution containing 10% pyridine and 1 mM 1,4-phenylene diisothiocyanate (PDITC) for 2 hours for surface activation. Chips are then rinsed with DMF and 1,2-dichloroethane and dried with Nitrogen.

The chip is then inserted in a solution of amine modified molecular beacon 1′M in a 1.0 M tris-HCL [pH 7.0] with 1% vol/vol N,N-diisopropylethylamine and 20% vol/vol dimethylsulfoxide (DMSO) buffer, and allowed to incubate overnight. Later chips are rinsed with Methanol and DI water and dried with nitrogen.

In order to prevent non-specific adsorption, the remaining unreacted PDITC are deactivated by immersing the chip in 50 mM 6-amino-1hexanol and 150 mM N,N-diisopropylethylamine in DMF for a minimum of 2 hours. Chips are then rinsed with DMF, Methanol and DI water and dried with nitrogen. After molecular beacon attachment, the chip is immersed in a solution containing the target DNA (5′-CACACACCAACCGTTGG-3′) with the 3′ end modified with ROX.

To obtain relatively high amplitude AC voltages an RF amplifier (EIN—Model 81 2100L-50 dB) is used in conjunction with a function generator (Agilent 33120A). Before heating the devices a droplet of buffer solution is dispensed to cover the devices without making contact with the probe needles. A 10 MHz AC bias is applied for 5 minutes for heating.

A Nikon Eclipse 600 fluorescence microscope may be used to image the surface of the chip. The fluorescein dye may be imaged using a DAPI filter and the ROX dye may be imaged using a TRITC filter. Images may be captured with a high resolution cooled CCD camera (Penguin 600CL).

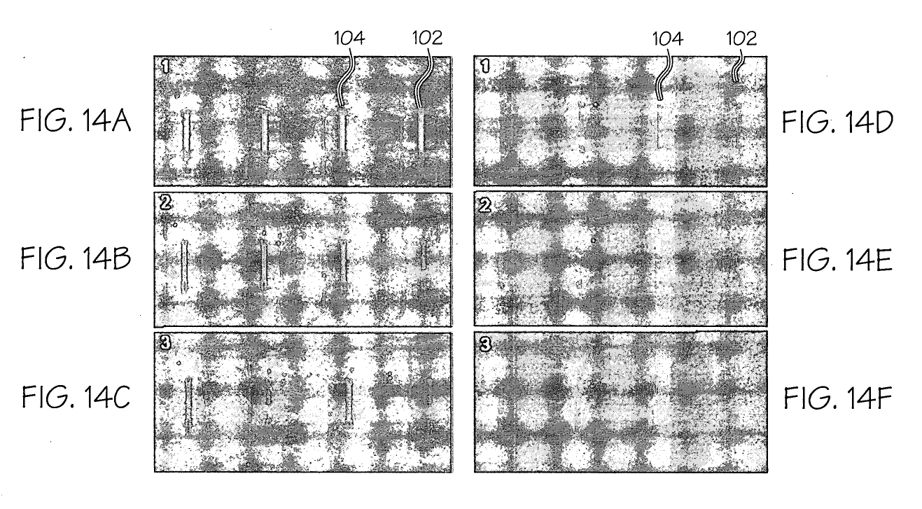

A comparison of fluorescence pictures of the devices before and after heating verifies the effect of heating on the functional coating on the device. In FIG. 14, the first column of images shows the complementary strand and the second column of images shows the molecular beacon fluorescence of the nanoplate devices. The first picture (1) in FIG. 14 shows four functionalized devices spaced with a 50′m pitch before the experiment. The fourth device from the left 102 was heated in fluid by applying an AC bias of 18.5 Vrms to the device for 5 minutes as described above. After heating, the chip was immediately immersed in a buffer solution for rinsing to wash away disassociated molecules, and was then dried with Nitrogen for Imaging. The results of this step are shown in the second picture (2) of FIG. 14. In both cases the fluorescence significantly decreased relative to unheated devices. A progressive decrease in the background intensity may be due to photobleaching during the experiments as time elapsed.

Later the second device from the left 104 was heated using the same procedure and was then imaged. The results of heating the second device from the left are shown in the third picture (3) of FIG. 14. Similarly, a significant decrease in the fluorescence in the heated device relative to unheated devices occurred. This decreased intensity suggests that either the hairpin loop is closing so that the fluorophore once again comes into contact with the internal quencher, or that the molecule itself is being removed. Upon re-immersing the chip into a buffer solution containing the complementary ROX modified DNA, neither fluorescence recovered. Therefore, it is believed that the molecular beacon and the complementary DNA detached from the surface.

Depending on which bonds are being broken for the detachment, the heated surfaces may be re-functionalized with new functional coatings. This will lead to a highly versatile and novel technique for individually functionalizing nanoscale sensor surfaces. By fine tuning the applied bias and heating the surface to 60±C, an exchange reaction may be performed without damaging the functional layer. The surface species may be characterized using energy dispersive x-ray spectroscopy (EDS).

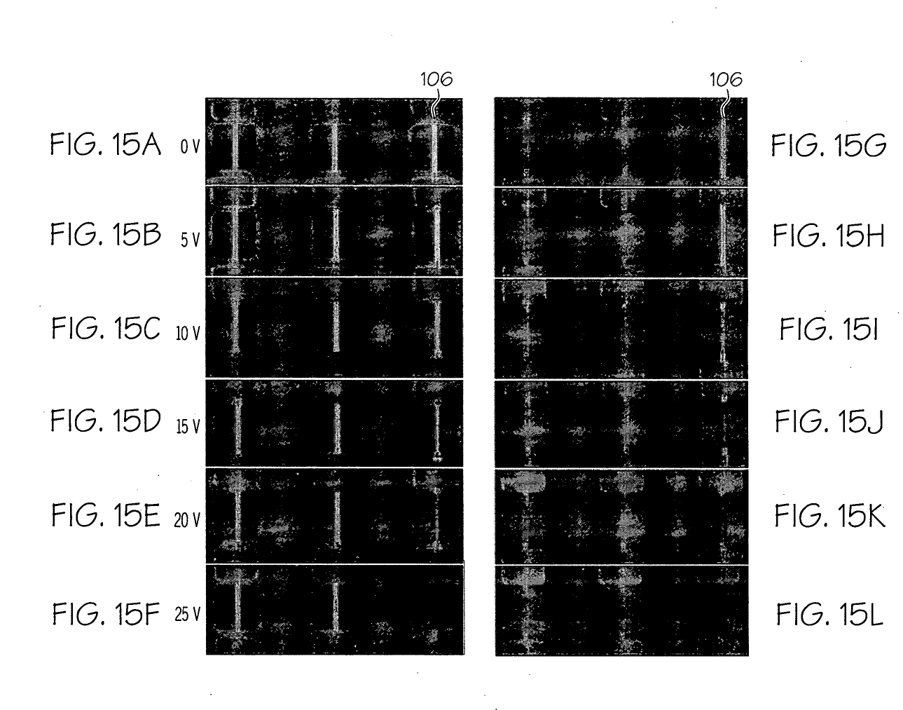

The fluorescence intensity of the heated devices may also be determined as a function of the applied voltage. Fluorescence images for the complementary strand (first column) and molecular beacon (second column) at different applied voltages are shown in FIG. 15. In FIG. 15, only the right most device 106 was heated, and the experiment was performed as outlined above. The decrease in the intensity may indicate the detachment of the molecules, and detachment is expected to occur only above a certain threshold voltage. As the applied voltage increases, the amount of observed fluorescence on the device decreased relative to the non-heated devices as shown in FIG. 15. The decrease was spatially non-uniform; i.e., intensity decreases were first noticed at the edges, then progressed to the center of the device with increasing voltage. Fringing fields may therefore play a role in the heating. Also, the fluid (which is a dielectric) may be directly being heated, due to the dielectric relaxation phenomena.

Control over the functionalized areas is desirable in producing high sensitivity detectors to prohibit competitive binding on non-active areas of the chip. Accordingly, the disclosed methods are designed to functionalize only the active areas to attain maximum sensitivity.

In the disclosed embodiments, the efficiency of the probe binding may successfully controlled by using silicon oxides with varying compositions. As the silicon content of the oxide increases, the attachment chemistry is less efficient.

Thermal oxide is a desirable surface on which to perform the immobilization chemistry. Thermal oxide has the chemical composition of SiOx where x is 2. Native oxide may be a less desirable surface on which to perform the chemistry, as no detectable attachment was observed to be present on the surface. A silicon rich PECVD oxide with x being less than 2 may be more efficient than native oxide but less efficient than thermal oxide.

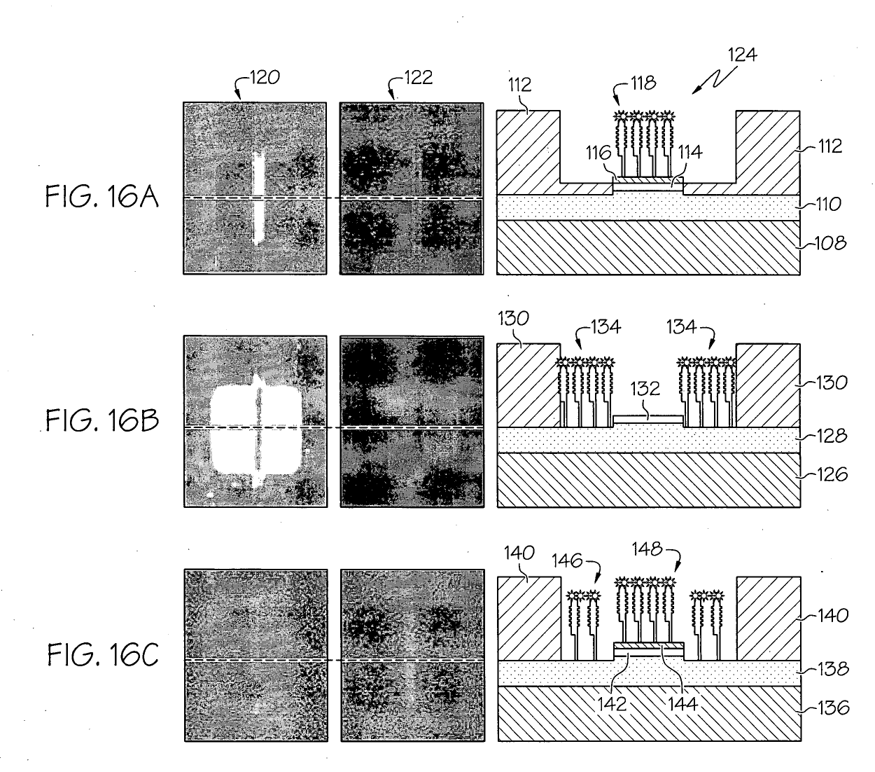

By patterning the device area accordingly, precise control over the functionalized area was achieved. FIG. 16 illustrates the degree of control that can be attained using this methodology. Each of rows A, B, and C of FIG. 16 show fluorescence images of the complementary strand 120 in the first column, fluorescence images of the beacon in the second column 122, and a schematic 124 of the device configuration in the third column.

In the first configuration of row A, the device includes silicon 108, buried oxide 110, PECVD oxide 112, silicon 114 and thermal oxide coating 116 of the nanoplate, and probe molecules 118 positioned on the nanoplate. In the second configuration of row B, the device includes silicon 126, buried oxide 128, PECVD oxide 130, nanoplate 132 and probe molecules 134 positioned on either side of the nanoplate. In the third configuration of row C of FIG. 16, the device includes silicon 136, buried oxide 138, PECVC oxide 140, silicon 142 and thermal oxide 144 of the nanoplate, probe molecules 146 positioned on either side of the nanoplate and probe molecules 148 positioned on the nanoplate.

Heating of individual nanoplate devices via dielectric heating using Liquid Crystals and fluorescent DNA molecules as described above provides localized heating in a fluidic environment. This allows individual functionalization of devices in an ultra large scale integrated device. Functional layers on devices with a pitch of 50′m are selectively removed, allowing new layers to be coated.

Also, by using silicon oxide films with varying silicon content, localized functionalization results in effective attachment chemistry only on the active areas of the devices. This allows specific regions to be functionalized with varying efficiencies using fully CMOS compatible materials with minor process variations.

One consideration in employing the disclosed method is that the surfaces of the heated devices need to be characterized subsequent to the heating step. An energy dispersive x-ray spectroscopy system integrated with a FESEM will meet the required resolution for accurate characterization of the surface. Using this technique, the silicon content of the surfaces can be obtained to determine the efficiency of the attachment chemistry on various types of oxide films before and after heating.

Depending on the molecules remaining on the surface after heating the devices, a number of approaches can be used for the re-functionalization of devices with new chemical species. Simply re-exposing the chip to the appropriate chemicals to build the necessary molecular levels may allow the re-attachment of distinct surface probes. This applies to oligonucleotide sequences but also should be applicable in the attachment of a wide variety of receptors for various biologically relevant macromolecules. The end result is a chip capable of the label-free sensing of a wide variety of biomolecules simultaneously in real time.

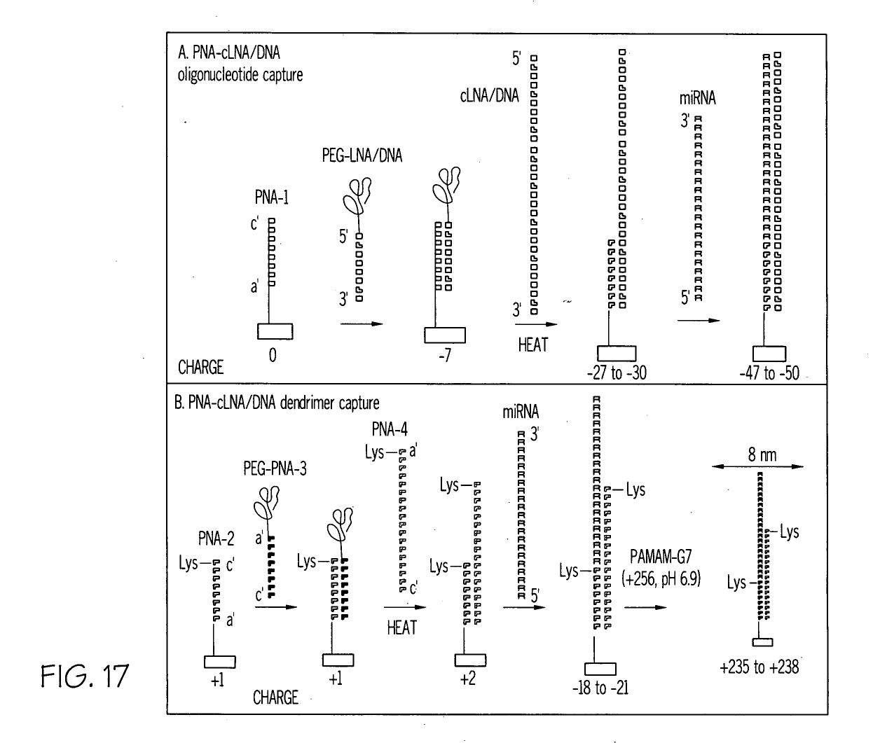

Another approach for locally functionalizing devices is to use a temperature mediated exchange reaction, for replacing a protective layer on the surface. In this scheme, initially a PNA probe is covalently attached to all of the devices. A complementary PNA strand modified with PEG(Poly Ethylene Glycol) is then be introduced as a capping molecule to all the devices. The PEG modification is desirable to prevent the non-specific binding of molecules in later steps. Later this protection layer is exchanged with the probe molecule by a heat mediated exchange reaction. This leads to the functionalization of only the heated devices, and allows for multiplexed detection. The technique is illustrated in FIG. 17. In FIG. 17(B), PAMAM-7 is introduced, which binds to the RNA to provide charge amplification.

Another concern with providing effective functionalization on a larger scale is reliable fabrication of the gate dielectric with a repeatable thickness. In the current fabrication scheme, all the oxide on top of the active area is etched, and native oxide is used as a gate dielectric. However, the surface chemistry may not attach efficiently on native oxide. There are a number of ways to alleviate these potential problems.

An etch stop may be placed on high quality thermal oxide. A thin deposited layer can be used as an etch stop on a thermally grown high quality gate oxide, which allows for the release of the devices without damaging the gate oxide. The etch stop can be removed selectively after opening the release windows, exposing the active area with a high quality thermal gate oxide.

Alternatively, a different surface chemistry may be used, i.e., one that results in molecules directly attached to silicon. Such sensors have been demonstrated to work effectively, and this may be used to provide a high quality gate oxide. Another approach is to use Atomic Layer Deposition (ALD) to deposit reliable thin dielectrics such as Aluminum Oxide after the release of the device. This dielectric can be functionalized using the above described chemistry.

Liftoff for passivation layer definition may also be used instead of etching, to allow the preservation of the high quality reliable gate oxide layer. The dielectric may be deposited by a number of methods including evaporation, sputtering or room temperature LPCVD. Doped silicon/polysilicon may be used as contacts and the passivation layer may be thermally grown while protecting the active area with Nitride. A metal that can be self-passivated as a contact may be used. For example, a metal such as Aluminum can be used as a contact metal, which later can be anodized to form a layer of passivation on the metal.

### Molecule Detection Systems

Sensing of a biomolecule using the above-described devices depends on the detection of a change in the conductance of the active area upon binding of the target analytes. This can be accomplished in multiple ways.

A bias can be applied between the source and drain contacts, and the gate bias can be adjusted such that the device is operating at the subthreshold region for maximum sensitivity. Measurements can be performed with any standard semiconductor parameter analyzer; by monitoring the source current versus time as the analyte is introduced, the presence of the analyte can be detected. A change in the current level indicates a binding event. The presence of the molecule and the magnitude of the change can then be directly correlated to the actual concentration of the analyte in the solution via use of appropriate control devices. By recording the change in conductance in real time the binding kinetics between the receptor and target molecules can be determined.

Applying a bias between the source and drain electrodes can cause problems due to the electric field setup in the fluid. Electrophoretic effects can cause molecules to move preferentially towards an electrode. Also, conduction through the fluid may become an issue as the bias is increased.

An alternative is to conduct an AC measurement in order to directly extract the conductance value at a 0 V DC bias. A sinusoidal signal produced by a function generator (Stanford Research Systems Model DS360) may be fed into the source electrode of the device. The resulting drain current may be amplified through a low-noise current preamplifier (Stanford Research Systems Model SR570) and detected using a lock-in amplifier (Stanford Research

Systems Model SR850). A switch matrix can be used for simultaneous measurement of multiple devices. The design will allow for the packaging of the device, so measurements can be performed in a robust fashion.

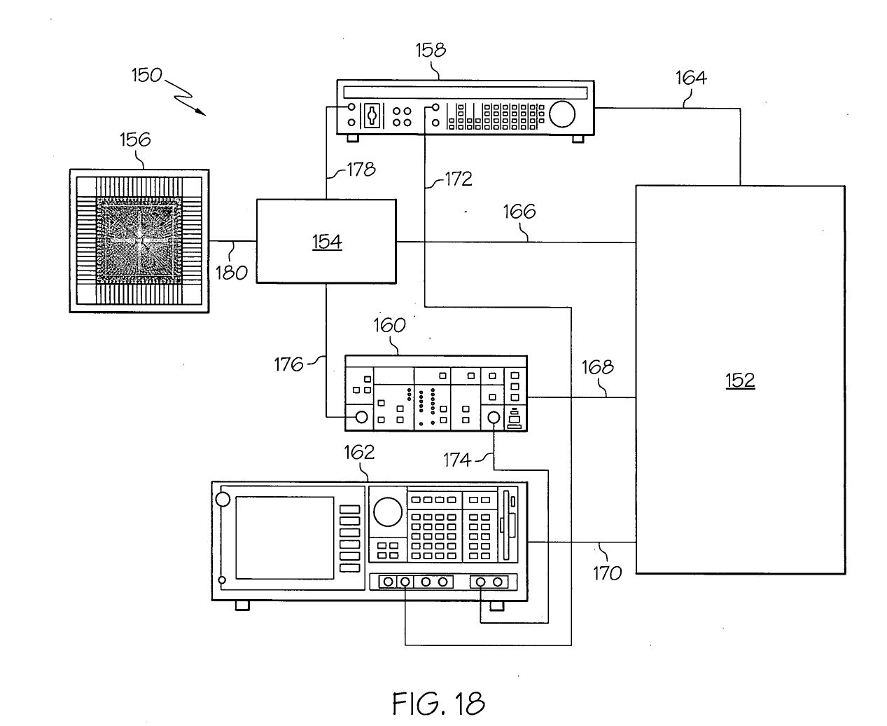

A schematic illustrating an embodiment of the above-described data acquisition system is shown in FIG. 18. A control computer or computing device 152 is electrically coupled to and communicates with switch matrix 154, function generator 158, preamplifier 160, and lock-in amplifier 162 via electrical signal conduits or links 164, 166, 168, 170, respectively.

Function generator 158 communicates with computer 152, amplifier 162 and switch matrix 154 via conduits or links 164, 172, 178 respectively. Switch matrix 154 communicates with function generator 158, computer 152, and preamplifier 160 via conduits or links 178, 166, 176 respectively. Switch matrix communicates with nanoplate device 156 via conduit or link 180. Preamplifier 160 communicates with computer 152, amplifier 162 and switch matrix 154 via conduits or links 168, 174, 176, respectively. Amplifier 162 communicates with computer 152, function generator 158, and preamplifier 160 via conduits or links 170, 172, 174 respectively.

Computer 152 includes processor hardware and software including executable programming instructions. The executable programs when executed cause the computer 152 to send and receive signals to and from the devices 158, 154, 160, 162, 156 to conduct measurements and operate the device as described above, and record or communicate the results.

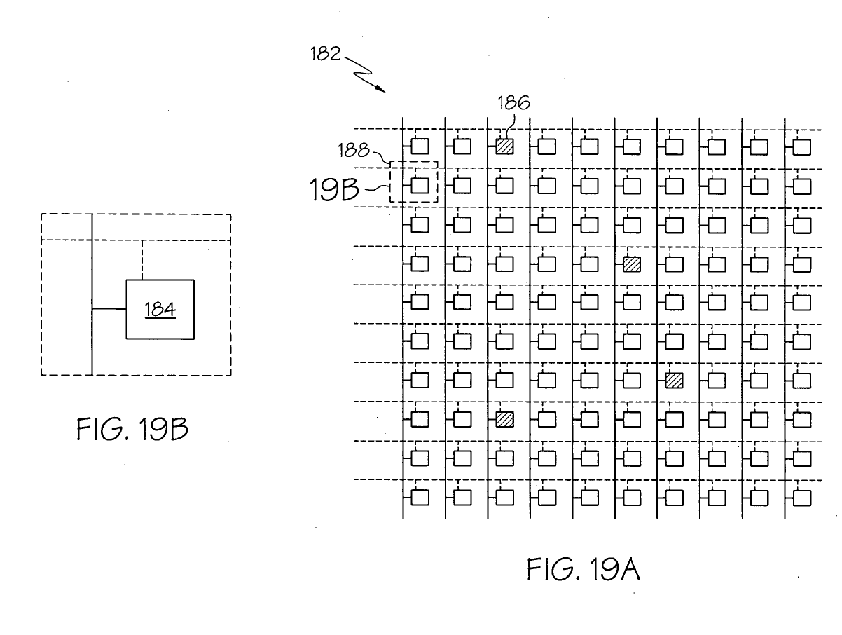

FIG. 19 is a simplified schematic of a platform for a nanosensor array 182, including a magnified view of a sensor site 184 which includes a nanosensor of one of the types described above. The array 182 may include nanowire or nanoplate sensors, or a combination of nanowire and nanoplates. In the illustrated embodiment, the array platform is a grid-TFT platform. Each sensor site 184 is individually addressable and as such can be individually heated or measured using row-column addressing. Each sensor site is functionalized with a specific receptor sequence. Site 186 and similarly darker-shaded sites represent individually heated, measured, or functionalized sites.

Row/column driver circuitry is used to address each sensor site, to increase the site temperature for the temperature-sensitive selective functionalization methods using bio-chemical receptor molecules described above. Once the sensor sites are functionalized with capture probes the conductivity map of the array system is recorded. Then, analytes are introduced into the system. Depending on the analyte sequences present, only the sensors with complementary receptor sequences are activated, with a corresponding modification in their conductance. A comparison of the before and after conductance maps allows parallel labelfree determination of the sequences present in the analyte.

The layout of the array 182 may be configured by calculating the inter-sensor pitch length. The sensor array is represented by SPICE-like compact electrical models with an equivalent circuit representation; and thermal and species transport are similarly represented by heat and mass transfer resistances. Self-consistent solution of the network determines the parasitic temperature rise in the neighboring cells due to heating of the selected sensor site. A system-level design approach reduces inadvertent heating and minimizes unintended functionalization of other sites in the array.

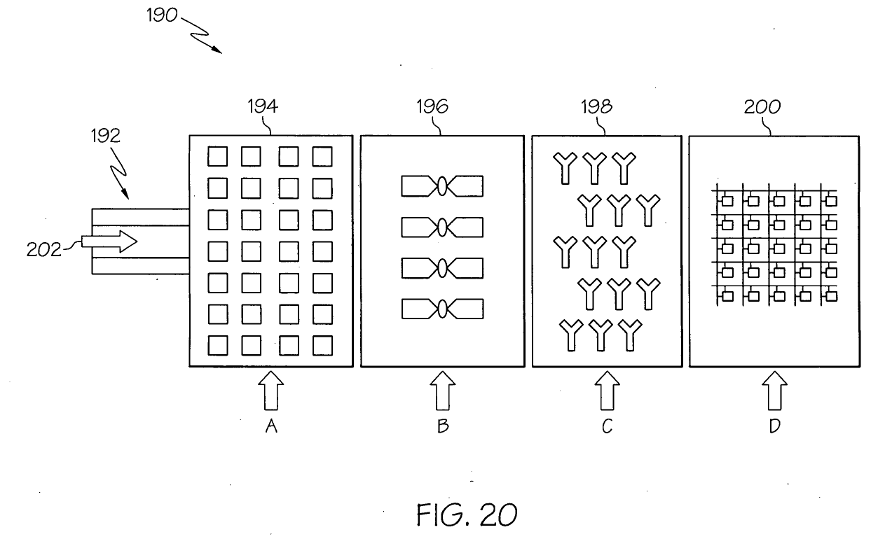

FIG. 20 is a simplified schematic of an integrated lab on a chip usable for detection of cancer proteins and markers, including a nanosensor array. The sensor platform 190 includes various modules 194, 196, 198, 200, each of which are configured to perform a particular function. In the illustrated example, analyte flows in the direction of arrow 202 through the fluidic port or ports 192. At (A), mechanical filtering occurs using an MEMS filter array 194. At (B), cell lysing occurs using an electrochemical lysing process 196. At (C), selective Ab or Abtomer capture is performed and at (D), electrical detection of the cancer proteins and/or markers is performed using a nanosensor array of one of the types described above.

In the illustrated embodiment, three biomarkers are used for prostrate cancer: PSA, H2 and CRISP-3. Folate receptor or folate binding protein can be used as a cancer marker. As such, folic acid is attached to the nanosensors via a silanized PEG linker for the detection of folate binding protein as a cancer marker.

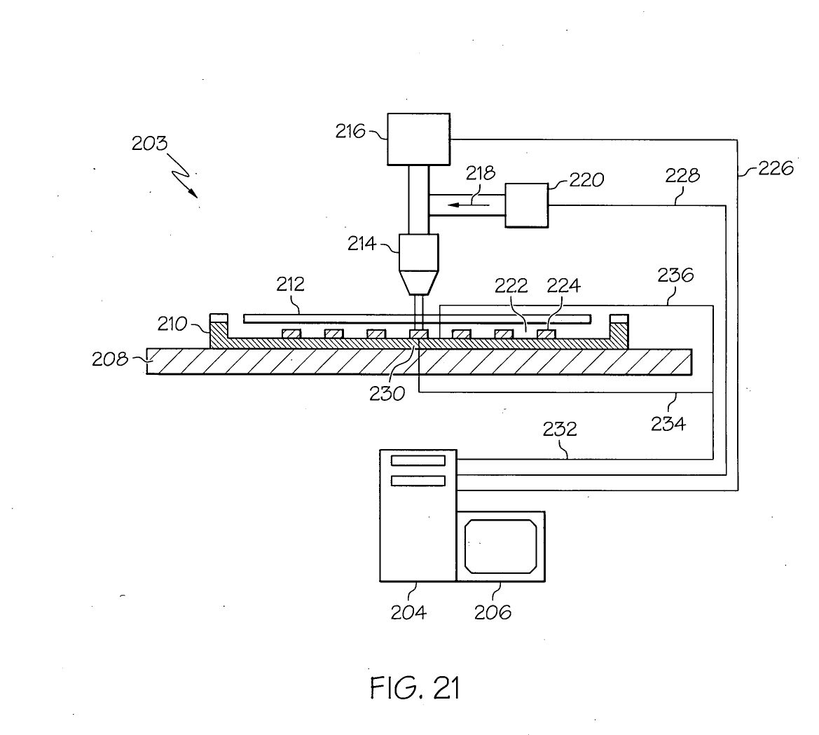

FIG. 21 is a simplified schematic of an embodiment of an apparatus for laser-mediated site-specific heating including a nanosensor array 203. The illustrated apparatus includes a computer or computing device 204, an output device (such as a digital display, LCD screen or computer monitor) 206, a stage 208, a substrate 210 (such as silicon) including a number of nanosensor devices 224, a cover (such as quartz) 212, a microscope 214, a CCD or similar camera 216, a laser source 220 generating a laser signal in the direction of arrow 218, a temperature sensor 230, and electrical or communication links 226, 228, 232, 234, 236 linking the system components to the computing device 204. Computing device 204 includes processor hardware and software configured to communicate with the system components and control the operation of the site-specific heating apparatus. The nanosensors 224 may be nanowires, nanoplates, or a combination thereof.

The nanosensor array is contained in a PDMS or glass covered enclosed microfluidic channel. The reagents flow through the channel and use the laser system to focus a beam on a nanosensor to locally raise the temperature and perform the LNA-PNA exchange reaction. The laser beam is focused by a lens to produce a heating spot diameter from a few microns to tens of microns. Since the laser energy absorbed by the water solvent is rapidly converted to heat due to the photo-thermal process, the heating volume is confined to the solution in the micro-channel just around the heating spot.

For programmable heating, computer 204 is used to control the laser power and pulse width. A Pt thin-film sensor is integrated in the device with probes very close to the heating point to measure the temperature at the heating spot inside the micro-channel, which is in turn used as the feedback by a LabView or similar program with automatic control algorithm to set the power voltage and pulse width for the laser source.

The stage 208 supporting the microchip also acts as a heat sink for fast a cooling step. A holium:YAG laser is used for the source since it emits at a wavelength of 2090 nm. Finite element simulation may be conducted using Fluent™ to optimize the inter-sensor distance and source power to achieve the required temperature profile. A similar strategy for linking different antibodies to specific array elements may be followed as described for the aptamers. However, because antibodies are more complex and require an attachment method that leaves the bound antibody active and accessible, a number of complementary methods of attachment may also be used.

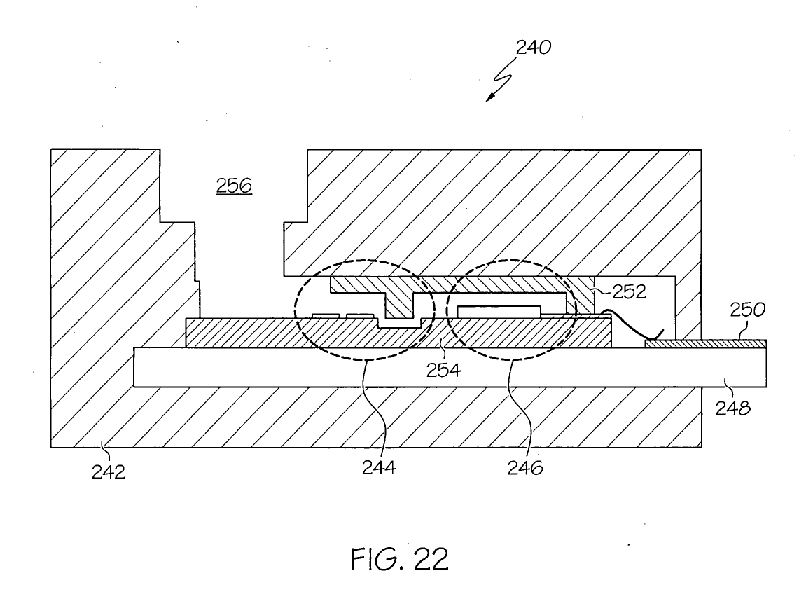

FIG. 22 is a representative schematic (not drawn to scale) of a disposable sensor cartridge including a nanosensor array. The integrated device 240 includes a multi-level cartridge 242 made of plastic or similar material, a filtration/lysing module (or integrated filter) 244, a nanosensor array 246, a PC board 248, an electrical interface 250, chip-level packaging 252, a microfluidic biochip 254, and a fluid sample port 256. The nanosensor array 246 may include nanowires, nanoplates or a combination thereof.

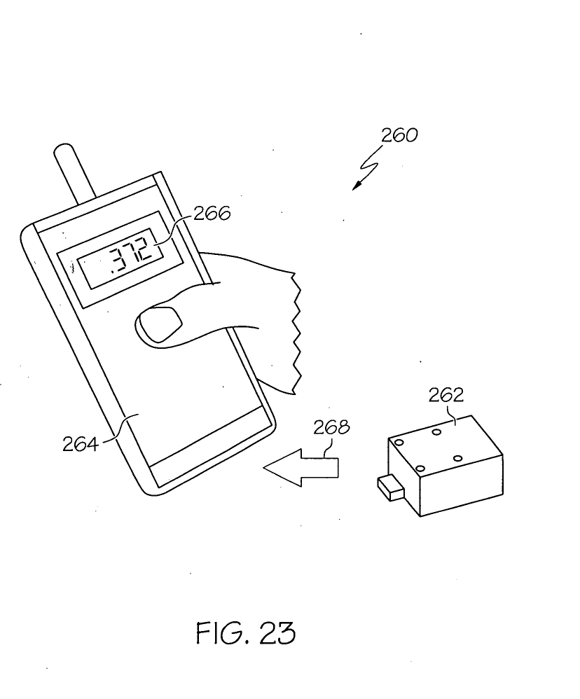

In the illustrated embodiment, the chips are attached to PC board 248 and wire bonded. The machined cartridge is configured to house the PC board with the chip, and also to provides a place to place the drop of fluid analyte (e.g. blood). The electronic hardware is configured to address the devices in the array (row and column addressing similar to a TFT screen), measure the resistance of the nanosensors, and perform the electrochemical lysing of the cells. The apparatus may be desktop sized, or, as shown in FIG. 23, portable.

FIG. 23 is a perspective view of an embodiment of a portable screening system including a measurement reader and a nanosensor cartridge. The system includes a handheld electronic impedance measurement reader 264 including a display 266, and a sensor cartridge 262. The cartridge is configured for one-time use. The display 266 is configured to display digital output, but may display text or graphics alternatively or in addition.

In prostate cancer applications involving detection of prostate cancer proteins and folate binding protein detection, the cells need to be separate from the blood sample; hence filters are integrated in the devices that trap cells and particulates larger than 1 um and let the rest of the fluid move to the functionalized sensor area. The nanowire and nanoplate sensor array is fabricated in silicon substrate with a PDMS cover and fluidic ports and integrated filter elements. Electrical measurements indicate the binding of proteins in a label free manner. The nanosensors are functionalized with antibodies, aptamers, or folic acid, as described above.

In breast cancer applications including breast cancer therapy monitoring, proteins from cell lysate needs to be detected as an indication of activation of signaling pathways. Hence the cells from the fluid extract are lysed. The cells are initially lysed externally and the lysate is introduced into the device. The cell lysing may also be performed in the device, using the electrochemical methods described. The cell lysate is then passed through the functionalized sensor array and signals are output.

Methods may be performed to determine the sensitivity limits of the fabricated devices. For example, the sensor response to buffer solutions at different pH values may be studied. Because the protonation/deprotonation of the surface hydroxyl groups with varying pH values changes the surface charge, the conductance of a device is expected to change with changing pH values.

Also, a method for sensing the presence of streptavidin in solution with devices functionalized with biotinylated Bovine Serum Albumin (BSA) may be performed. BSA attaches to oxide surfaces by electrostatic interactions; hence, the functionalization scheme is relatively straightforward. Also, the biotin-streptavidin pair has been shown to have a very high binding affinity, which is ideal for testing the devices. With appropriate controls, this method enables selective sensing of biomolecules. Detection is performed with varying concentrations of streptavidin to determine the sensitivity limit of the devices.

In addition, a method for detection of a relatively short DNA molecule (12 base pairs long) may be performed by using covalently immobilized DNA or PNA(Peptide Nucleic Acid) probes on the surface. Sensing is performed by introducing target molecules at varying concentrations, in order to determine the sensitivity of the devices. This method provides the capability of biomolecular sensing using covalently immobilized probe molecules, and the sensitivity of the devices can be compared to results in the literature.

To perform multiplexed real time detection of biomolecules, selective functionalization of the sensor devices is combined with sensing methods described above. For example, label-free multiplexed detection of micro RNA(miRNA) is performed using the above-described techniques. Such a method is of particular interest because there are currently no known label-free detection methods for miRNA. An miRNA is a short strand of non-coding RNA with about 21-23 nucleotides, that interacts with the messenger RNA (mRNA) to downregulate gene expression. It has been shown that expressions of certain miRNA are directly related to cancer, and levels of expression correlate to the state of various diseases.

In the detection method, composite PNA(Peptide Nucleic Acid)-LNA(Locked Nucleic Acid) probes are used for selective sensing of miRNA molecules due to the high binding affinity between these molecules. For example, label-free multiplexed detection of miRNA may be performed for one or more of the sequences that are shown in Table 1.

TABLE 1

| | |
|---|---|
| miRNA | Sequence (5′-3′) |

| | |
|---|---|
| hsa-miR-1 | UGG AAU GUA AAG AAG UAU GUA |
| hsa-miR-7 | UGG AAG ACU GUG AUU UUG UU |
| hsa-miR-124a | UUA AGG CAC GCG GUG AAU GCC A |
| hsa-miR-134 | UGU GAC UGG UUG ACC AGA GGG |
| hsa-miR-193a | AAC UGG CCU ACA AAG UCC CAG |
| hsa-miR-193b | AAC UGG CCC UCA AAG UCC CGC UUU |

The nanoplate devices and methods described above may be used as diagnostic and screening tools, as well as for other applications. For example, to study intracellular biomolecules, cells can be captured on the nanosensor device and can be easily lysed by flowing DI water.

The described devices and methods may also be used to screen miRNA from cells. The living cells can flow into the chip, and can be captured either by micromachined mechanical filters or dielectrophoretic filters. After the capture, cells are lysed simply by flowing DI water to expose the sensors to the cell lysate. By screening for multiple miRNA molecules the oncology of the cell can be studied to identify whether a cancer is present and if so, the type of cancer present.

Methods can also be performed with multiple cells, and the communication between cells can be intercepted using the devices by screening for multiple analytes simultaneously. For example, the effect of certain drugs on the response behavior of the cells can be studied in detail, again by trapping cells, and introducing various drug molecules.

Further, the idea of site selective heating of silicon devices can be used beyond just site specific functionalization. This approach has the following additional uses, among others.

A method is provided to use micro and nanoscale spatial control at specific sites to perform large scale parallel ‘PCR on a pixel’ within a large array of devices. This is also referred to as ‘PCR on a transistor’ or ‘PCR on a nanowire’. According to the presently disclosed method, the magnitude of the AC voltage is adjusted to obtain the desired temperature and perform very localized heating and amplification of target molecules and each pixel/wire/transistor that is being heated is controlled and optimized individually. For example, a single bacteria or virus is trapped on a transistor and then lysed by heating the transistor to 95 C and then temperature-cycled to perform PCR (polymerase chain reaction) locally and an increase in fluorescence can be observed.

Besides PCR, localized heating of devices can be used as means to carry out various reactions sensitive to heat in close proximity, enabling heat selective chemical reactions. Fundamentally, temperature is a variable for all chemical reactions, and the rate of any chemical reaction can be controlled in high spatial and temporal resolution, owing to the localized heating and low thermal mass associated with the method.

The disclosed heating methods may also be used to change the surface properties of the sensors, for example, from hydrophobic to hydrophilic or vice versa, by the local heating. This can also be used to transport fluids or particles from device to device.

Alternatively, the amplified molecules are detected using label-free electronic means using impedance or charge-sensing approaches such as the same field effect transistor used for the heating and temperature generation.

A method is also provided for the local control of temperature, which allows creation of temperature gradients that can be used to move fluid droplets or fluid or particles with different properties within a medium. The change in temperature changes the surface tension and results in movement of droplets. According to this method, a linear array of devices is sequentially heated and particles or droplets of interest are moved using this change in surface tension (also referred to as Marangoni Effect), which is control by change in temperature. This movement of fluid is designed to be very useful in a wide variety of microfluidic lab-on-a-chip platforms, especially if chemical reactions like lysing, PCR and other steps are also used in the same platform, along with electrical detection.

The local temperature control may also be used to check for stringency of DNA hybridization at each pixel, since the local temperature can be increased and precisely controlled and strands that are not bound as tightly as others can be denatured. This may be done in micro-arrays. According to this method, the entire chip is heated and heating of individual sites may or may not be able to be controlled.

The present disclosure describes patentable subject matter with reference to certain illustrative embodiments. Variations, alternatives, and modifications to the illustrated embodiments may be included in the scope of protection available for the patentable subject matter.

What is claimed is:

1. A method of forming a sensor array having a number of sensors configured to detect at least one target molecule in a liquid analyte, the method comprising:
exposing the sensor array to a liquid containing a probe molecule,
applying, in the presence of the liquid containing the probe molecule, an AC signal to one sensor of the sensor array to heat the one sensor, and
binding the probe molecule to the one sensor.

2. The method of claim 1, wherein each sensor includes a nanodevice.

3. The method of claim 2, wherein each nanodevice includes a semiconductor, an insulator, at least one metal contact, and a microfluidic component.

4. The method of claim 3, wherein each nanodevice is fabricated by a top-down, CMOS-compatible process.

5. The method of claim 3, where each nanodevice is fabricated by a bottoms-up self-assembly technique.

6. The method of claim 5, wherein each nanodevice is fabricated using a bonded Silicon on Insulator wafer using a Separation by Implantation of Oxygen technique.

7. The method of claim 6, wherein a first liquid is used to apply a first probe molecule and a second liquid different than the first liquid is used to apply a second probe molecule different than the first probe molecule.

8. The method of claim 7, wherein the exposing, binding and applying steps are repeated to form a sensor configured to detect a second target molecule different than the target molecule.

9. The method of claim 1, wherein the exposing, binding and applying steps are applied to a first sensor in the sensor array to configure the first sensor to detect a first target molecule and the exposing, binding, and applying steps are applied to a second sensor in the sensor array to configure the second sensor to detect a second target molecule different than the first target molecule.

10. The method of claim 1, wherein a first AC signal having a first voltage is applied to a first sensor in the sensor array to configure the first sensor and a second AC signal having a second voltage different than the first voltage is applied to a second sensor in the sensor array to configure the second sensor.

11. The method of claim 1, wherein the applying step heats a first sensor in the sensor array to a first temperature value and heats a second sensor in the sensor array to a second temperature value different than the first temperature value to configure the sensor array.

12. The method of claim 1, wherein the AC signal is applied to the sensor between an electrode and a bottom backgate of the sensor.

13. A method of detecting at least one target molecule in a liquid analyte with a sensor array having a number of sensors, the method comprising:
exposing the sensor array to a liquid containing a probe molecule,
applying, in the presence of the liquid containing the probe molecule, an AC signal to one sensor of the sensor array to heat the one sensor,
binding the probe molecule to the one sensor,
exposing the sensor array to the liquid analyte,
applying an AC signal to the one sensor of the sensor array to heat the one sensor and the probe molecule, and
detecting the at least one target molecule with the probe molecule, if the target molecule is present in the liquid analyte.

14. The method of claim 13, wherein the sensor array is formed according to the method of claim 1.

15. A molecule detection apparatus comprising:
a nanoplate sensor device made by the method of claim 1, and
a computer memory including software logic executable to perform the method of claim 13.

16. The apparatus of claim 15, comprising a function generator coupled to the nanoplate sensor to provide an electrical signal to a sensor of the nanoplate sensor device.

17. The apparatus of claim 16, wherein the electrical signal is a sinusoidal signal.

18. The apparatus of claim 17, comprising a switch matrix coupled to the sensor and to the function generator to enable simultaneous measurement of multiple sensors.

19. A cancer screening system including the apparatus of claim 15.

20. An miRNA screening system including the apparatus of claim 15.

21. A method of selectively heating individual sites in a nanosensor array including a plurality of nanosensors, the method comprising:
determining a desired pitch length between sensors in the array,
arranging the sensors in the array according to the desired pitch length,
individually addressing each sensor site in the sensor array,
determining a desired temperature for a sensor site in the array, and
selectively increasing the temperature of the sensor site to the desired temperature by applying an AC voltage to the sensor site.

22. A method for performing a temperature acceleratable chemical reaction according to claim 21.

23. A method of lysing cells according to claim 21.

24. The method of claim 23, wherein the method is used to detect intracellular proteins.

25. The method of claim 21, wherein the determining and selectively increasing steps are repeated to heat the sensor site to a different desired temperature.

26. A method of performing a polymerase chain reaction at an individual sensor site, according to claim 25.

27. A method of changing surface properties of a nanosensor device, according to claim 25.


SEQUENCE LISTING (8 sequences)

SEQ ID NO: 1 - DNA, 22 bp, Artificial Sequence
  Description of Artificial Sequence Synthetic oligonucleotide
ccaacggttggtgtgtggttgg

SEQ ID NO: 2 - DNA, 17 bp, Artificial Sequence
  Description of Artificial Sequence Synthetic oligonucleotide
cacacaccaaccgttgg

SEQ ID NO: 3 - RNA, 21 nt, Homo sapiens
uggaauguaaagaaguaugua

SEQ ID NO: 4 - RNA, 20 nt, Homo sapiens
uggaagacugugauuuuguu

SEQ ID NO: 5 - RNA, 22 nt, Homo sapiens
uuaaggcacgcggugaaugcca

SEQ ID NO: 6 - RNA, 21 nt, Homo sapiens
ugugacugguugaccagaggg

SEQ ID NO: 7 - RNA, 21 nt, Homo sapiens
aacuggccuacaaagucccag

SEQ ID NO: 8 - RNA, 24 nt, Homo sapiens
aacuggcccucaaagucccgcuuu